(19) United States
(12) Patent Application Publication
(10) Pub. No.: US 2023/0248777 A1
(43) Pub. Date: Aug. 10, 2023

(54) AUGMENTATION OF FIBROBLAST CELL THERAPY EFFICACY BY MICROBIOME MANIPULATION

(71) Applicant: FIGENE, LLC, Houston, TX (US)

(72) Inventor: Thomas ICHIM, San Diego, CA (US)

(21) Appl. No.: 17/999,588

(22) PCT Filed: May 29, 2021
(86) PCT No.: PCT/US2021/070642

§ 371 Date: Nov. 22, 2022

Related U.S. Application Data
(60) Provisional application No. 63/031,810, filed on May 29, 2020.

(51) Int. Cl. A61K 35/33; C12N 5/077; A61K 35/742; A61K 35/745; A61P 29/00

(52) U.S. Cl. CPC A61K 35/33; A61K 35/742; A61K 35/745; A61P 29/00; C12N 5/0656; C12N 2501/15; C12N 2501/599

(57) ABSTRACT

Disclosed are means, methods, and compositions of matter useful for enhancing therapeutic efficacy of fibroblasts, fibroblast products such as exosomes, or fibroblast apoptotic bodies, through modulation of the recipient microbiome prior to, concurrent with, and subsequent to administration of fibroblasts. In one embodiment, assessment of dysbiosis is performed prior to administration of fibroblast therapy, and dysbiosis is repaired by means including: a) microbiome transplant; b) prebiotic treatment; and/or c) probiotic treatment.

This application claims priority to U.S. Provisional Pat. Application Serial No. 63/031,810, filed May 29, 2020, which is incorporated by reference herein its entirety.

### TECHNICAL FIELD

Embodiments of the disclosure include at least the fields of cell biology, molecular biology, physiology, biology, and medicine. More specifically, the present disclosure concerns manipulation of dysbiosis in an individual prior to treatment of a medical condition with fibroblasts.

### BACKGROUND

The human microbiome consists of 10-100 trillion symbiotic microbial cells harbored in the human body, predominantly in the human intestinal tract. The bacteria, archaea, and eukarya colonizing the gastrointestinal tract are collectively called the gut microbiome. Over the past decade, the microbiome has emerged as a critical component in the regulation of host health. Microbes perform many processes that the human body cannot handle itself. For example, microbes digest food to generate nutrients for host cells, synthesize vitamins, metabolize drugs, detoxify carcinogens, stimulate renewal of cells in the gut lining and activate and support the immune system.

Emerging evidence suggests that the microbiota play a role in the pathogenesis of many diseases and disorders. Dysbiosis in the gastrointestinal (GI) microbiome is associated with various GI disorders such as C. difficile colitis, inflammatory bowel diseases (IBD) such as ulcerative colitis (UC) and Crohn’s disease, irritable bowel syndrome (IBS), Celiac disease, and general antibiotic-associated microbial dysbiosis. There is also evidence that antibiotic treatment reduces the efficacy of regenerative medicine. Therefore, manipulation of the microbiome may be crucial for the treatment of these GI disorders.

Fibroblasts have been demonstrated to possess a number of regenerative properties. The present disclosure satisfies a long-felt need in the art for effective methods and compositions to restore biosis, a state of microbiome health, to increase the therapeutic activity of regenerative fibroblasts.

### BRIEF SUMMARY

The present disclosure is directed to compositions and methods for repairing dysbiosis, for example as a conditioning step prior to administration of fibroblast-based cellular therapy. In some embodiments, fibroblasts, modified fibroblasts, fibroblast exosomes, and/or fibroblast conditioned media are utilized as a means of treating degenerative, immunological, or inflammatory conditions in a patient subsequent to repair of the microbiome.

In one embodiment, provided is method of increasing the efficacy of a fibroblast-based treatment comprising the steps of: a) treating dysbiosis in an individual in need thereof; and b) administering the fibroblast-based treatment, wherein treating the dysbiosis can occur prior to, at the same time as, and/or after administering the fibroblast-based treatment. The method may further comprise the steps of: identifying the individual in need thereof; and assessing dysbiosis in said individual.

In one embodiment of the method, the fibroblast-based treatment comprises a fibroblast-based cellular treatment. The fibroblast-based cellular treatment can comprise fibroblast cells which are allogeneic, autologous, or xenogenic to the recipient. The fibroblast cells may be plastic adherent. The fibroblast cells may possess regenerative activity, which can include but is not limited to stimulation of angiogenesis, inhibition of inflammation, enhancement of differentiation activity, suppression of Th17, enhancement of M2 and T regulatory cell generation, augmentation of tissue self-renewal, or a combination thereof. The fibroblast cells may express CD105, CD73, and/or FGF-2 and may inhibit production of IL-18, macrophage activation, and/or mast cell activation. The fibroblast cells may also produce IL-10. In some cases, the fibroblasts are preconditioned by treatment with oxytocin, which can be at a concentration of 1-100 IU or 10 IU per ml of tissue culture media.

In another embodiment of the method, the fibroblast-based treatment comprises a fibroblast-conditioned culture media treatment. Administration of the fibroblast-conditioned culture media may induce generation of CD8 cells. In some cases, the fibroblast-conditioned culture media can comprise at least about 5 ng/ml or 2 ng/ml of fibroblast-produced FGF-2. IN some cases, the fibroblast-conditioned culture media comprises at least about 20 pg/ml of fibroblast-produced TGF-β. The fibroblast-conditioned culture media may also comprise exosomes derived from fibroblasts, which can express CD81 and/or membrane-bound TNG-β, for example. The exosomes can be at least about 60-200 nm in size.

In some embodiments of the method, dysbiosis is assessed by ribosomal sequencing. The dysbiosis can comprise an abnormality in the microbiome of the host, which can include commensal bacteria in the gut, blood, skin, epithelial tissue, bone marrow, thymus, spleen, liver, or a combination thereof. In some embodiments, the dysbiosis results from a genetic predisposition to dysbiosis. In some embodiments, the dysbiosis is induced by a treatment, which can comprise antibiotics, chemotherapy, radiation therapy, immunotherapy, hormonal therapy, or a combination thereof. Dysbiosis can be treated by administration of microbiome components from an external source, such as, for example, fecal transplant. Dysbiosis can also be treated by administration of prebiotics, probiotics, or a combination thereof. Prebiotics can include soluble starch, yeast extract, polydextrose, polydextrose powder, lactulose, lactosucrose, raffinose, gluco-oligosaccharide, inulin, fructo-oligosaccharide, isomalto-oligosaccharide, soybean oligosaccharides, lactosucrose, xylo-oligosaccharide, chito-oligosaccharide, manno-oligosaccharide, aribino-oligosaccharide, siallyl-oligosaccharide, fuco-oligosaccharide, galacto-oligosaccharide, gentio-oligosaccharides, or a combination thereof. Probiotics can include Bifidobacterium spp., Lactobacillus spp., Streptococcus thermophilia, Bacillus coagulans, Bacillus laterosporus, Pediococcus acidilactici, Saccharomyces boulardii, or a combination thereof.

In some embodiments, the fibroblast-based treatment of the method is for stroke. In some embodiments, the fibroblast-based treatment of the method is for degenerative disc disease. In some embodiments, the fibroblast-based treatment of the method is for spinal cord injury. In some embodiments, the fibroblast-based treatment of the method is for liver failure. In some embodiments, the fibroblast-based treatment of the method is for autism. In some embodiments, the fibroblast-based treatment of the method is for chronic inflammation, which can be due to stent placement.

Also provided in one embodiment is a composition for treating dysbiosis comprising prebiotics, probiotics, or a combination thereof, wherein the composition further comprises a fibroblast-based treatment. The prebiotics of the composition can include soluble starch, yeast extract, polydextrose, polydextrose powder, lactulose, lactosucrose, raffinose, gluco-oligosaccharide, inulin, fructo-oligosaccharide, isomalto-oligosaccharide, soybean oligosaccharides, lactosucrose, xylo-oligosaccharide, chito-oligosaccharide, manno-oligosaccharide, aribino-oligosaccharide, siallyl-oligosaccharide, fuco-oligosaccharide, galacto-oligosaccharide, gentio-oligosaccharides, or a combination thereof. The probiotics of the composition can comprise Bifidobacterium spp., Lactobacillus spp., Streptococcus thermophilia, Bacillus coagulans, Bacillus laterosporus, Pediococcus acidilactici, Saccharomyces boulardii, or a combination thereof.

In some embodiments, dysbiosis is assessed by ribosomal sequencing prior to, at the same time as, and/or after administering the composition. The dysbiosis can comprise an abnormality in the microbiome of the host, which can include commensal bacteria in the gut, blood, skin, epithelial tissue, bone marrow, thymus, spleen, liver, or a combination thereof. In some embodiments, the dysbiosis results from a genetic predisposition to dysbiosis. In some embodiments, the dysbiosis is induced by a treatment, which can comprise antibiotics, chemotherapy, radiation therapy, immunotherapy, hormonal therapy, or a combination thereof. Dysbiosis can be treated by administration of microbiome components from an external source, such as, for example, fecal transplant. Dysbiosis can also be treated by administration of prebiotics, probiotics, or a combination thereof.

In one embodiment, the fibroblast-based treatment of the composition is a fibroblast-based cellular treatment. The prebiotics and/or probiotics can be administered prior to, at the same time as, or after the fibroblast-based cellular treatment. The fibroblast-based cellular treatment comprises fibroblast cells which are allogeneic, autologous, or xenogenic to the recipient and may be plastic adherent. The fibroblast cells may possess regenerative activity, which can include but is not limited to stimulation of angiogenesis, inhibition of inflammation, enhancement of differentiation activity, suppression of Th17, enhancement of M2 and T regulatory cell generation, augmentation of tissue self-renewal, or a combination thereof. The fibroblast cells may express CD105, CD73, and/or FGF-2 and may inhibit production of IL-18, macrophage activation, and/or mast cell activation. The fibroblast cells may also produce IL-10. In some cases, the fibroblasts are preconditioned by treatment with oxytocin, which can be at a concentration of 1-100 IU or 10 IU per ml of tissue culture media.

In another embodiment, the fibroblast-based treatment of the composition is a fibroblast-conditioned culture media treatment. The prebiotics and/or probiotics can be administered prior to, at the same time as, or after the fibroblast-conditioned culture media treatment. Administration of the composition containing the fibroblast-conditioned culture media may induce generation of CD8 cells. In some cases, the fibrob last-conditioned culture media can comprise at least about 5 ng/ml or 2 ng/ml of fibroblast-produced FGF-2. IN some cases, the fibroblast-conditioned culture media comprises at least about 20 pg/ml of fibroblast-produced TGF-β. The fibroblast-conditioned culture media may also comprise exosomes derived from fibroblasts, which can express CD81 and/or membrane-bound TNF-β, for example. The exosomes can be at least about 60-200 nm in size.

In some embodiments, the fibroblast-based treatment of the composition is for stroke. In some embodiments, the fibroblast-based treatment of the composition is for degenerative disc disease. In some embodiments, the fibroblast-based treatment of the composition is for spinal cord injury. In some embodiments, the fibroblast-based treatment of the composition is for liver failure. In some embodiments, the fibroblast-based treatment of the composition is for autism. In some embodiments, the fibroblast-based treatment of the composition is for chronic inflammation, which can be due to stent placement.

It is specifically contemplated that any limitation discussed with respect to one embodiment of the disclosure may apply to any other embodiment of the disclosure. Furthermore, any composition of the disclosure may be used in any method of the disclosure, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Summary, Detailed Description, Claims, and Description of Figure Legends.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIG. 1 shows Disease Activity Index scores for an inflammatory bowel disease mouse model at days 3, 5, and 7 post-treatment. Group 1 mice were treated with dextran sodium sulfate (DSS) (control); group 2 mice were treated with DSS and fibroblasts; group 3 mice were treated with DSS and Bifidobacterium infantis; and group 4 mice were treated with DSS, Bifidobacterium infantis, and fibroblasts. n = 10 per group. From left to right, the bars are Control; DSS plus Bifidobacterium infantis; DSS and fibroblasts; and DSS and Bifidobacterium infantis and fibroblasts.

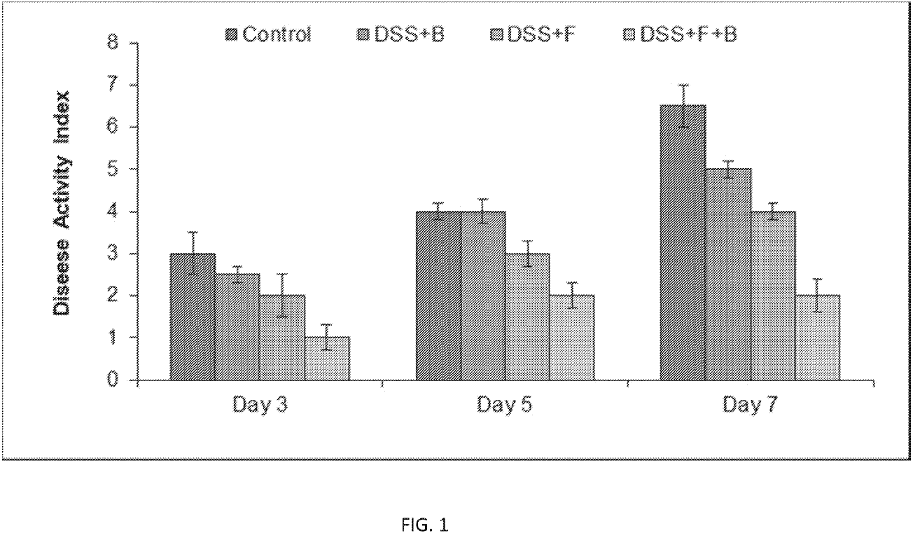

FIG. 2 shows quantified Interleukin-6 (IL-6) levels for an inflammatory bowel disease mouse model at days 3, 5, and 7 post-treatment. Group 1 mice were treated with dextran sodium sulfate (DSS) (control); group 2 mice were treated with DSS and fibroblasts; group 3 mice were treated with DSS and Bifidobacterium infantis; and group 4 mice were treated with DSS, Bifidobacterium infantis, and fibroblasts. n = 10 per group. From left to right, the bars are Control; DSS plus Bifidobacterium infantis; DSS and fibroblasts; and DSS and Bifidobacterium infantis and fibroblasts.

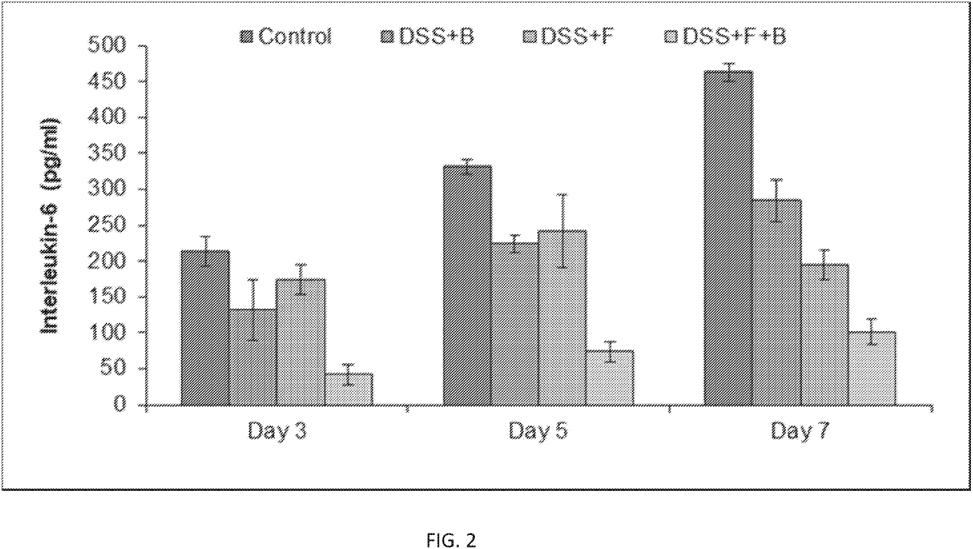

FIG. 3 shows that fibroblast immunotherapy of multiple sclerosis is enhanced by antibiotic manipulation of the microbiome. From left to right, the bars are Control, Antibiotic, Fibroblasts alone, and Antibiotic plus Fibroblasts.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc.., as well as individual numbers within that range, for example, 1, 2, 3, 4, 5, and 6. This applies regardless of the breadth of the range. When ranges are present, the ranges may include the range endpoints.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, “allogeneic” refers to tissues or cells or other material from another body that in a natural setting are immunologically incompatible or capable of being immunologically incompatible, although from one or more individuals of the same species.

“C. difficile” refers to the bacterium Clostridium difficile. C. difficile is a spore-forming bacterium that is especially prevalent in soil. C. difficile causes debilitating and sometimes deadly colitis in humans and is the leading cause of antibiotic-associated diarrhea. The primary symptom of C. difficile infection (CDI) is watery diarrhea, which also acts as the primary mode of transmission. It is estimated that C. difficile caused approximately 453,000 infections and was associated with 29,000 deaths in the US in 2011, with the highest incidence in individuals 65 years or older, whites, and/or females. The recommended treatments for CDI are vancomycin or fidaxomicin, but these antibiotics are not effective for every patient, and recurrent infection is common in some patients.

As used herein, “cell culture” means conditions wherein cells are obtained (e.g., from an organism) and grown under controlled conditions (“cultured” or grown “in culture”) outside of an organism. A primary cell culture is a culture of cells taken directly from an organism (e.g., tissue cells, blood cells, cancer cells, neuronal cells, fibroblasts, etc.). Cells are expanded in culture when placed in a growth medium under conditions that facilitate cell growth and/or division. The term “growth medium” means a medium sufficient for culturing cells. Various growth media may be used for the purposes of the present disclosure including, for example, Dulbecco’s Modified Eagle Media (also known as Dulbecco’s Minimal Essential Media) (DMEM), or DMEM-low glucose (also DMEM-LG herein). DMEM-low glucose may be supplemented with fetal bovine serum (e.g., about 10% v/v, about 15% v/v, about 20% v/v, etc.), antibiotics, antimycotics (e.g., penicillin, streptomycin, and/or amphotericin B), and/or 2-mercaptoethanol. Other growth media and supplementations to growth media are capable of being varied by the skilled artisan. The term “standard growth conditions” refers to culturing cells at 37° C. in a standard humidified atmosphere comprising 5% CO₂. While such conditions are useful for culturing, it is to be understood that such conditions are capable of being varied by the skilled artisan who will appreciate the options available in the art for culturing cells. When cells are expanded in culture, the rate of cell proliferation is sometimes measured by the amount of time needed for the cells to double in number. This is referred to as doubling time.

As used herein, “cell line” refers to a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to seeding density, substrate, medium, growth conditions, and time between passaging.

“CFU,” as used herein, stands for colony forming unit and refers to the amount of bacteria in a probiotic that are viable and capable of dividing and forming bacterial colonies.

“Clostridium butyricum” is a butyrate-producer and known member of the healthy human gut microbiome. “Faecalibacterium prausnitzii” is one of the most important groups of butyrate-producers in the healthy human gut. “Roseburia faecis” and “Roseburia intestinalis” are butyrate-producing bacteria. “Eubacterium limosum” is a member of the healthy human gut microbiome.

“Colonic butyrogenic bacteria” refer to intestinal bacteria that produce butyrate as a byproduct of fermentation. They are gram-positive Firmicutes with high phylogenetic diversity. The most abundant groups of butyrate producers include Eubacterium rectale, Roseburia spp., and Faecalibacterium prausnitzii.

The term “compatible,” as used herein, means that the components of pharmaceutical compositions are capable of being commingled with the cells and/or conditioned media of the present disclosure and/or with other components of the pharmaceutical compositions, in a manner such that the desired pharmaceutical efficacy is not substantially impaired.

As used herein, “conditioned medium” describes medium in which a specific cell or population of cells has been cultured for a period of time, and then removed, thus separating the medium from the cell or cells. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. In this example, the medium containing the cellular factors is conditioned medium.

As used herein, “dysbiosis” refers to a microbial imbalance on or within the body. More specifically, dysbiosis can refer to a microbial imbalance within the gut microbiome.

“Differentiation” (e.g., cell differentiation) describes a process by which an unspecialized (or “uncommitted”) or less specialized cell acquires the features (e.g., gene expression, cell morphology, etc.) of a specialized cell, such as a nerve cell or a muscle cell for example. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. In some embodiments of the disclosure, “differentiation” of fibroblasts to other cell types is described. This process may also be referred to as “transdifferentiation”.

As used herein, “dedifferentiation” refers to the process by which a cell reverts to a less specialized (or less committed) position within the lineage of a cell. As used herein, the lineage of a cell defines the heredity of the cell. The lineage of a cell places the cell within a hereditary scheme of development and differentiation. Within the context of the current disclosure, “dedifferentiation” may refer to fibroblasts acquiring more “immature” associated markers such as OCT4, NANOG, CTCFL, ras, raf, SIRT2 and SOX2. Additionally, “dedifferentiation” may mean acquisition of functional properties such as enhanced proliferation activity and/or migration activity towards a chemotactic gradient. In some embodiments fibroblasts may be “dedifferentiated” by treatment with various conditions, subsequent to which they are “differentiated” into other cell types.

“Fibroblasts” refer to a cell, progenitor cell, or differentiated cell and include isolated fibroblast cells or population(s) thereof capable of proliferating and differentiating into ectoderm, mesoderm, or endoderm. In some embodiments, fibroblasts are utilized in an autologous or allogenic manner. As used herein, placental and adult-derived cellular populations are included in the definition of “fibroblasts.” In the context of the present disclosure, fibroblast cells may be derived through means known in the art from sources including at least foreskin, ear lobe skin, bone marrow, cord blood, placenta, amnion, amniotic fluid, umbilical cord, embryos, intraventricular cells from the cerebral spinal fluid, circulating fibroblast cells, mesenchymal stem cell associated cells, germinal cells, adipose tissue, exfoliated tooth derived fibroblasts, hair follicle, dermis, skin biopsy, nail matrix, parthenogenically-derived fibroblasts, fibroblasts that have been reprogrammed to a dedifferentiated state, side population-derived fibroblasts, fibroblasts from plastic surgery-related by-products, and the like.

The term “fibroblast-derived product” (also “fibroblast-associated product” or “fibroblast-produced therapeutic factor”), as used herein, refers to a molecular or cellular agent derived or obtained from one or more fibroblasts. In some cases, a fibroblast-derived product is a molecular agent. Examples of molecular fibroblast-derived products include conditioned media from fibroblast culture, microvesicles obtained from fibroblasts, exosomes obtained from fibroblasts, apoptotic vesicles obtained from fibroblasts, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

As used herein, “gut flora,” “gastrointestinal flora,” “intestinal flora,” “gut microbiome,” “intestinal microbiome,” “microbiome,” and the like are interchangeable and are intended to represent the normal, naturally occurring bacterial population present in the gastric and intestinal systems of healthy humans and animals. The terms are meant to reflect both the variety of bacterial species and the concentration of bacterial species found in a healthy human or animal.

As used herein, “gut,” “intestine,” “intestinal tract,” “colon,” and the like are used interchangeably and are intended to represent the gastrointestinal system of humans.

“Pharmaceutically acceptable carrier,” as used herein, includes one or more compatible solid or liquid filler diluents or encapsulating substances that are suitable for administration to a human or other animal. In the present disclosure, the term “carrier” thus denotes an organic or inorganic ingredient, which can be natural or synthetic, and with which the molecules of the disclosure are combined to facilitate application or administration. The carrier must also be compatible with the agent used to produce a desired result or exert a desired influence on the particular condition being treated.

As used herein, “prebiotic” refers to a selectively fermented ingredient that induces specific changes to the composition and/or activity of gastrointestinal microflora to confer benefits upon host well-being and health. Examples of prebiotics include but are not limited to inulin, arabinoxylan, xylose, soluble fiber dextran, soluble corn fiber, polydextrose, lactose, N-acetyl-lactosamine, glucose, galactose, fructose, rhamnose, mannose, uronic acids, 3′-fucosyllactose, 3′-sialylactose, 6′-sialyllactose, lacto-N-neotetraose, 2′-2′-fucosyllactose, trans-galactooligosaccharides, glucooligosaccharides, isomaltooligosaccharides, lactosucrose, polydextrose, soybean oligosaccharides, and arabinose, cellobiose, fructose, fucose, galactose, glucose, lactose, lactulose, maltose, mannose, ribose, sucrose, trehalose, xylobiose, xylooligosaccharide, D-xylose, and/or xylitol.

As used herein, “probiotic” refers to a composition containing at least one live probiotic bacterial strain. “Probiotics” are live bacteria or yeast that, when consumed, confer a health benefit to the host. Probiotics are said to restore the balance of bacteria in the gut after disruption due to long-term antibiotic use or gastrointestinal disease. Examples of probiotics include but are not limited to Bifidobacterium spp., Lactobacillus spp., Streptococcus thermophilia, Bacillus coagulans, Bacillus laterosporus, Pediococcus acidilactici, and/or Saccharomyces boulardii.

“RCDI,” as used herein, stands for recurrent C. difficile infection. RCDI includes patients with 2 or more incidences of CDI after discontinuation of antibiotic therapy. There are several antibiotics that have been used successfully in treatment of CDI including metronidazole, vancomycin, and fidaxomicin. However, in RCDI patients, these antibiotics only partially resolve CDI symptoms and recurrence of CDI after the antibiotic discontinuation. Fecal microbiota transplantation (FMT), however, has been shown to be a highly effective method of treating RCDI.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, “regenerative activities” include but are not limited to therapeutic functions, stimulation of angiogenesis, inhibition of inflammation, and/or augmentation of tissue self-renewal, for example in part through activation of endogenous and/or exogenous stem and/or progenitor cells. In one embodiment, treatment of dysbiosis is used to augment the regenerative activities of fibroblasts. Regenerative activities include the promotion of angiogenesis; enhancement of differentiation efficacy, for example, if under regular conditions 100 fibroblasts cultured in HGF resulted in differentiation of 25 hepatocytes, enhanced differentiation would result in differentiation of more than 25 hepatocytes; suppression of inflammation; enhanced ability to generate T regulatory cells; increased ability to create M2 macrophages; and increased ability to suppress Th17 production. In a further embodiment, fibroblasts having regenerative activities are isolated for one or more specific markers and subsequently transfected with genes capable of endowing various therapeutic functions. Genes useful for stimulation of regenerative activities such as augmentation of hematopoietic activity include interleukin (IL)-12 and IL-23 to stimulate proliferation of hematopoietic stem cells, for example. Other useful genes include IL-35, wherein IL-35 transfection allows for generation of cells possessing anti-inflammatory and angiogenic T regulatory cell activity, and the cells possessing T regulatory cell activities include cells expressing the transcription factor FoxP3, as an example.

As used herein, “short chain fatty acids” (SCFA) refer to a group of fatty acids with less than six carbons that are produced by anaerobes of the human large intestine. Certain types of gut bacteria ferment indigestible polysaccharides, resulting in the production of three major SCFAs: acetate, propionate, and butyrate. SCFAs are a major source of energy not only for enterocytes but also for the entire body. In fact, it is estimated that 60-75% of the energy derived from ingested carbohydrates can be attributed to SCFA production. In addition to their contribution to host metabolism, SCFAs also influence colonic health through regulation of epithelial proliferation and differentiation. “Butyrate” is a SCFA considered to play an important role in the regulation of digestive health, though the connection between the gut microbiome and this crucial SCFA only recently made. Colonic epithelial cells prefer butyrate as a food source. It is estimated that 70% to 90% of produced butyrate is metabolized by colonocytes, and overall, the colonic epithelium obtains 60-70% of its required energy from butyrate.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition related to bone. For subjects having or suspected of having a medical condition directly or indirectly associated with bone, the medical condition may be of one or more types. The subject may have a disease or be suspected of having the disease. The subject may be asymptomatic. The subject may be of any gender. The subject may be of a certain age, such as at least 30, 35, 40, 45, 50, 55, 60, 65, 70, 75, 80, 85, 90, 95, or 100 or more.

As used herein, the phrase “subject in need thereof” or “individual in need thereof” refers to a subject or individual, as described infra, that suffers or is at a risk of suffering (e.g, pre-disposed such as genetically pre-disposed, or subjected to environmental conditions that pre-dispose, etc.) from the diseases or conditions listed herein (e.g, dysbiosis).

“Synbiotic” refers to a composition that contains both a prebiotic and a probiotic.

As used herein, the term “therapeutically effective amount” is synonymous with “effective amount”, “therapeutically effective dose”, and/or “effective dose” refers to an amount of an agent sufficient to produce a desired result or exert a desired influence on the particular condition being treated. In some embodiments, a therapeutically effective amount is an amount sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. Effective amount can also mean the amount of a compound, material, or composition comprising a compound of the present disclosure that is effective for producing some desired effect, e.g., treating or preventing ovarian failure. For instance, in some embodiments, the effective amount refers to the amount of fibroblasts and/or fibroblast-conditioned media that can augment manipulation of the microbiome and/or treat and/or prevent dysbiosis. The effective amount may vary depending on the organism or individual treated.

The appropriate effective amount to be administered for a particular application of the disclosed methods can be determined by those skilled in the art, using the guidance provided herein. For example, an effective amount can be determined experimentally using various techniques and/or extrapolated from in vitro and in vivo assays including dose escalation studies. Various concentrations of an agent may be used in preparing compositions incorporating the agent to provide for variations in the age of the patient to be treated, the severity of the condition, and/or the duration of the treatment and the mode of administration. One skilled in the art will recognize that the condition of the individual can be monitored throughout the course of therapy and that the effective amount of a compound or composition disclosed herein that is administered can be adjusted accordingly. Further, one of skill in the art recognizes that an amount may be considered effective even if the medical condition is not totally eradicated but improved partially. For example, the medical condition may be halted or reduced or its onset delayed, a side effect from the medical condition may be partially reduced or completed eliminated, and so forth.

As used herein, the terms “treatment,” “treat,” or “treating” refers to intervention in an attempt to alter the natural course of the individual or cell being treated, and may be performed either for prophylaxis or during the course of pathology of a disease or condition, such as for example ovarian failure. Treatment may serve to accomplish one or more of various desired outcomes, including, for example, preventing occurrence or recurrence of disease, alleviation of symptoms, and diminishment of any direct or indirect pathological consequences of the disease, preventing disease spread, lowering the rate of disease progression, amelioration or palliation of the disease state, and remission or improved prognosis, and/or producing some desired effect, e.g. augmentation of manipulation of the microbiome and/or treatment and/or prevention of dysbiosis.

As used herein, a “trophic factor” describes a substance that promotes and/or supports survival, growth, proliferation and/or maturation of a cell. Alternatively or in addition, a trophic factor stimulates increased activity of a cell. The interaction between cells via trophic factors may occur between cells of different types. Cell interaction by way of trophic factors is found in essentially all cell types, and is a particularly significant means of communication among various types of cell types. Trophic factors also can function in an autocrine fashion, i.e., a cell may produce trophic factors that affect its own survival, growth, differentiation, proliferation, and/or maturation. Factors produced by fibroblasts are selected from HGF, FGF-1. FGF-2, FGF-5, CTNF, PDGF-BB, EGF, and/or TGF-β. Growth factors include one or more of GM-CSF, IL-15, IL-1a, IL-2, IL-4, IL-5, IL-6, IL-7, IL-8, MCP-1, TNFα, FGF-2, Flt-3, PDGF-AA, PDGF-BB, TGF-β1, TGF-β2, and TGF-β3.

### I. Preparation of Regenerative Fibroblasts

Certain aspects of the present disclosure relate to the preparation of regenerative fibroblast cells for treatment of individuals after dysbiosis is corrected. Regenerative fibroblast cells may be generated by culturing fibroblasts under sufficient conditions to generate a regenerative fibroblast cell. In some embodiments, the fibroblast cells can provide a tissue with regenerative activity. In some embodiments, the method includes culturing the population of fibroblast regenerative cells under conditions that support proliferation of the cells. In additional embodiments, the fibroblast cells may be cultured under conditions that form tissue aggregate bodies. In some embodiments, the fibroblast cells are used to create other cell types for tissue repair or regeneration. Generation of fibroblasts has been described previously in the art and is incorporated herein. Generally, fibroblasts are harvested, for example, biopsied, from dissociated tissues of interest. Accomplishing the dissociation process can be performed mechanically or by treatment of tissue with enzymes, such as dispase or collagenase, which releases cells from the tissues. Subsequently, single cell fibroblasts are isolated based on plastic adherence and then allowed to expand and proliferate in liquid media. Fibroblasts can be purified into subset populations, such as those expressing CD73 or CD105, or can be utilized in an unpurified state.

Specific desirable properties of fibroblast cells of the present disclosure are the ability to increase endothelial function; suppress inflammation; induce neo-angiogenesis; prevent atrophy; differentiate into functional tissue; induce local resident stem and/or progenitor cells to proliferate through secretion of soluble factors or membrane bound activities; induce generation of T regulatory cells; suppress generation of Th17 cells; and/or enhance the generation of M2 macrophages. The fibroblasts may also inhibit macrophage and/or mast cell activation. In one embodiment, fibroblast cells are collected from an autologous patient, expanded ex vivo, and reintroduced into the patient at a concentration and frequency sufficient to cause therapeutic benefit. The fibroblast cells are selected for the ability to cause neo-angiogenesis, prevent tissue atrophy, regenerate functional tissue, induce proliferation, induce generation of T regulatory cells, suppress generation of Th17 cells, and/or enhance the generation of M2 macrophages.

When selecting fibroblast cells, several factors must be taken into consideration, including the ability for ex vivo expansion without loss of therapeutic activity, ease of extraction, general potency of activity, and potential for adverse effects. Ex vivo expansion ability of fibroblasts can be measured using typical proliferation and colony assays known to one skilled in the art, for example, while identification of therapeutic activity may utilize one or more functional assays that test biological activities correlated with one or more therapeutic goals.

In some embodiments, assessment of therapeutic or regenerative activity is performed using surrogate assays which detect one or more markers associated with a specific therapeutic activity. In some embodiments, assays used to identify therapeutic activity of fibroblast cell populations include evaluation of the production of one or more factors associated with desired therapeutic activity. In some embodiments, evaluation of the production of one or more factors to approximate therapeutic activity in vivo includes identification and quantification of the production of FGF, VEGF, angiopoietin, other angiogenic molecules, PDGF-BB, interleukin 1 receptor antagonist, soluble HLA-G, or a combination thereof, which may be used to serve as a guide for approximating therapeutic activity in vivo.

In some embodiments, the regenerative fibroblast cells used in combination with a probiotic/enzyme mixture to treat dysbiosis are collected from an autologous patient, expanded ex vivo, and reintroduced into the patient at a concentration and frequency sufficient to cause therapeutic benefit. In other cases, the expanded cells are allogeneic or xenogenic with respect to a recipient individual. In some embodiments, fibroblasts of the present disclosure are used as precursor cells that differentiate following introduction into an individual. In some embodiments, fibroblasts are subjected to differentiation into a different cell type (e.g., a hematopoietic cell) prior to introduction into the individual. The fibroblasts may be preconditioned with oxytocin, which can increase the therapeutic activity of the fibroblasts. The oxytocin may be administered at a concentration of, for example, 1-100 IU or 10 IU per ml of culture media in which the fibroblasts are cultured.

Compositions of the present disclosure may be obtained from isolated fibroblast cells or a population thereof capable of proliferating and differentiating into ectoderm, mesoderm, or endoderm. In some embodiments, the enriched population of fibroblast cells are about 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, or 11-12 micrometers in size. The fibroblasts may be plastic adherent.

In some embodiments, the fibroblast cells are expanded in culture using one or more cytokines, chemokines and/or growth factors prior to administration to an individual in need thereof. The agent capable of inducing fibroblast expansion can be selected from the group consisting of TPO, SCF, IL-1, IL-3, IL-7, flt-3L, G-CSF, GM-CSF, Epo, FGF-1, FGF-2, FGF-4, FGF-20, VEGF, activin-A, IGF, EGF, NGF, LIF, PDGF, a member of the bone morphogenic protein family, and a combination thereof. The agent capable of inducing fibroblast differentiation can be selected from the group consisting of HGF, BDNF, VEGF, FGF1, FGF2, FGF4, FGF20, and a combination thereof.

In some embodiments, the fibroblast cells express proteins characteristic of normal fibroblasts including the fibroblast-specific marker, CD90 (Thy-1), a 35 kDa cell-surface glycoprotein, and the extracellular matrix protein, collagen. In some embodiments, an isolated fibroblast cell expresses at least one of Oct-4, Nanog, Sox-2, KLF4, c-Myc, Rex-1, GDF-3, LIF receptor, CD105, CD117, CD344, or Stella markers. In some embodiments, the fibroblasts express CD73, CD90, and/or CD105. In some embodiments, fibroblast cells are isolated and expanded and possess one or more markers selected from a group consisting of CD10, CD13, CD44, CD73, CD90, CD141, PDGFr-alpha, HLA-A, HLA-B, HLA-C, or a combination thereof. In some embodiments, fibroblasts of the present disclosure express telomerase, Nanog, Sox2, β-III-Tubulin, NF-M, MAP2, APP, GLUT, NCAM, NeuroD, Nurr1, GFAP, NG2, Olig1, Alkaline Phosphatase, Vimentin, Osteonectin, Osteoprotegrin, Osterix, Adipsin, Erythropoietin, SM22-α, HGF, c-MET, α-1-Antriptrypsin, Ceruloplasmin, AFP, PEPCK 1, BDNF, NT-⅘, TrkA, BMP2, BMP4, FGF2, FGF4, PDGF, PGF, TGFα, TGFβ, and/or VEGF. In some embodiments, the fibroblast cells do not produce one or more of CD14, CD31, CD34, CD45, CD117, CD141, HLA-DR, HLA-DP, HLA-DQ, or a combination thereof. In further embodiments, the fibroblast regenerative cell has enhanced expression of GDF-11 as compared to a control. In still further embodiments, the fibroblast cells express CD73, which is indicative of fibroblast cells having regenerative activity.

In some embodiments, an isolated fibroblast cell does not express at least one of MHC class I, MHC class II, CD45, CD13, CD49c, CD66b, CD73, CD105, or CD90 cell surface proteins. Such isolated fibroblast cells may be used as a source of conditioned media. In some embodiments, the method optionally includes the step of depleting cells expressing stem cell surface markers or MHC proteins from the cell population, thereby isolating a population of stem cells. In some embodiments, the cells to be depleted express MHC class I, CD66b, glycophorin a, or glycophorin b.

In some cases, fibroblast cells are obtained from a biopsy, and the donor providing the biopsy may be either the individual to be treated (autologous), or the donor may be different from the individual to be treated (allogeneic). In some embodiments, the fibroblast cells are xenogenic with respect to a recipient individual. In some embodiments wherein allogeneic fibroblast cells are utilized for an individual, the fibroblast cells may come from one or a plurality of donors. In some embodiments fibroblasts are used from young, pre-pubescent donors. In some embodiments, steps are taken to protect allogeneic or xenogenic cells from immune-mediated rejection by the recipient. Steps include encapsulation, co-administration of an immune suppressive agent, transfection of said cells with immune suppressory agent, or a combination thereof. In some embodiments, tolerance to the cells is induced through immunological means. In some embodiments, fibroblasts are transfected with genes to allow for enhanced growth and to overcome the Hayflick limit. Subsequent to derivation by biopsy, the fibroblasts are expanded in culture using standard cell culture techniques.

Biopsy is performed by extracting tissue, usually 0.1 grams to 10 grams. In some situations biopsy may be performed using a biopsy gun or alternatively using a scalpel. In one embodiment, biopsies are from skin tissue (dermis and epidermis layers) from a subject’s post-auricular area. In one embodiment, the starting material is composed of three 3-mm punch skin biopsies collected using standard aseptic practices, though the methods disclosed herein for preparing a skin biopsy tissue would apply equally to biopsies of other tissue types. The biopsies are collected by the treating physician, placed into a vial containing sterile phosphate buffered saline (PBS), and stored at 2-8° C. Upon initiation of the process, biopsies are inspected, and accepted biopsy tissues are washed prior to enzymatic digestion. After washing, a Liberase Digestive Enzyme Solution is added without mincing, and the biopsy tissue is incubated at 37.0 ± 2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Other commercially available collagenases may also be used, such as Serva Collagenase NB6 (Helidelburg, Germany).

After digestion, Iscove’s Complete Growth Media (IMDM, GA, 10% Fetal Bovine Serum (FBS)) is added to neutralize the enzyme, and cells are pelleted by centrifugation and re-suspended in 5.0 mL IMDM. Alternatively, full enzymatic inactivation is achieved by adding IMDM without centrifugation. Additional IMDM is added prior to seeding of the cell suspension into a particular flask (such as a T-175 cell culture flask) for initiation of cell growth and expansion. Alternatively, a T-75, T-150, T-185, or T-225 flask can be used in place of the T-175 flask. Cells are incubated at 37.0 ± 2° C. with 5.0 ± 1.0% CO₂ and supplemented with fresh IMDM every three to five days by removing half of the IMDM and replacing it with the same volume with fresh media. Alternatively, full IMDM replacements can be performed.

In specific cases, cells are not cultured in the T-175 flask for more than 30 days prior to passaging. Confluence is monitored throughout the process to ensure adequate seeding densities upon culture splitting. When cell confluence is greater than or equal to 40% in the T-175 flask, the cells are passaged by removing the spent media, washing the cells, and treating the cells with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF), or a Two Layer Cell Stack (2 CS) can be used in place of the T-500 Flask.

Morphology may be evaluated at each passage and prior to harvest to monitor culture purity throughout the process by comparing the observed sample with visual standards for morphological examination of cell cultures. Typical fibroblast morphologies when growing in cultured monolayers include elongated, fusiform, or spindle-shaped cells with slender extensions or larger, flattened stellate cells with cytoplasmic leading edges. A mixture of these morphologies may also be observed. Fibroblasts in less confluent areas can be similarly shaped but randomly oriented. The presence of keratinocytes in cell cultures may also be evaluated. Keratinocytes are round and irregularly shaped and, at higher confluence, appear to be organized in a cobblestone formation. At lower confluence, keratinocytes are observable in small colonies.

Cells are incubated at 37 ± 2° C. with 5.0 ± 1.0% CO₂ and passaged every three to five days for cells in T-500 flasks and every five to seven days for cells in ten layer cell stacks (10CS). Cells should not be cultured for more than 10 days prior to passaging. When cell confluence in a T-500 flask is ≥95%, cells are passaged to a 10 Layer Cell Stack (10 CS) culture vessel. Alternately, two Five Layer Cell Stacks (5 CS) or a 10 Layer Cell Factory (10 CF) can be used in place of the 10 CS. Passage to the 10 CS is performed by removing the spent media, washing the cells, and treating with Trypsin-EDTA to release adherent cells in the flask into the solution. Cells are then transferred to the 10 CS. Additional IMDM is added to neutralize the trypsin, and the cells from the T-500 flask are pipetted into a 2 L bottle containing fresh IMDM.

The contents of the 2 L bottle may be transferred into the 10 CS and seeded across all layers of the 10 CS. Cells are then incubated at 37.0 ± 2° C. with 5.0 ± 1.0% CO₂ and supplemented with fresh IMDM every five to seven days. In some cases, cells should not be cultured in the 10 CS for more than 20 days prior to passaging. In one embodiment, the passaged fibroblasts are rendered substantially free of immunogenic proteins present in the culture medium by incubating the expanded fibroblasts for a period of time in protein-free medium. When cell confluence in the 10 CS is ≥95%, cells are harvested. Harvesting is performed by removing the spent media, washing the cells, treating with Trypsin-EDTA to release adherent cells into the solution, and adding additional IMDM to neutralize the trypsin. Cells are collected by centrifugation and resuspended, and quality control testing is performed to determine total viable cell count and cell viability as well as sterility and the presence of endotoxins.

In some embodiments, the fibroblast dosage formulation is a suspension of fibroblasts obtained from a biopsy using standard tissue culture procedures. The cells in the formulation display typical fibroblast morphologies when growing in cultured monolayers. Specifically, cells may display an elongated, fusiform or spindle appearance with slender extensions, or cells may appear as larger, flattened stellate cells which may have cytoplasmic leading edges. A mixture of these morphologies may also be observed. The cells may also express proteins characteristic of normal fibroblasts, including the fibroblast-specific marker CD90 (Thy-1), a 35 kDa cell-surface glycoprotein, and the extracellular matrix protein collagen.

In one embodiment, regenerative fibroblast cells are purified from cord blood. Cord blood fibroblast cells are fractionated, and the fraction with enhanced therapeutic activity is administered to the patient. In some embodiments, cells with therapeutic activity are enriched based on physical differences (e.g., size and weight), electrical potential differences (e.g., charge on the membrane), differences in uptake or excretion of certain compounds (e.g., rhodamine-123 efflux), as well as differences in expression marker proteins (e.g., CD73). Distinct physical property differences between stem cells with high proliferative potential and low proliferative potential are known. Accordingly, in some embodiments, cord blood fibroblast cells with a higher proliferative ability are selected, whereas in other embodiments, a lower proliferative ability is desired. In some embodiments, cells are directly injected into the area of need and should be substantially differentiated. In other embodiments, cells are administered systemically and should be less differentiated, so as to still possess homing activity to the area of need.

In embodiments where specific cellular physical properties are the basis of differentiating between cord blood fibroblast cells with various biological activities, discrimination on the basis of physical properties can be performed using a Fluorescent Activated Cell Sorter (FACS), through manipulation of the forward scatter and side scatter settings. Other embodiments include methods of separating cells based on physical properties using filters with specific size ranges, density gradients, and pheresis techniques. In embodiments where differentiation is based on electrical properties of cells, techniques such as electrophotoluminescence are used in combination with a cell sorting means such as FACS. In some embodiments, selection of cells is based on ability to uptake certain compounds as measured by the ALDESORT system, which provides a fluorescent-based means of purifying cells with high aldehyde dehydrogenase activity. Without being bound by theory, cells with high levels of this enzyme are known to possess higher proliferative and self-renewal activities in comparison to cells possessing lower levels. Further embodiments include methods of identifying cells with high proliferative activity by identifying cells with ability to selectively efflux certain dyes such as rhodamine-123, Hoechst 33342, or a combination thereof. Without being bound to theory, cells possessing this property often express the multidrug resistance transport protein ABCG2 and are known for enhanced regenerative ability compared to cells which do not possess this efflux mechanism.

In some embodiments, cord blood cells are purified for certain therapeutic properties based on the expression of markers. In one particular embodiment, cord blood fibroblast are purified for cells with the endothelial precursor cell phenotype. Endothelial precursor cells or progenitor cells expressing markers such as but not limited to CD133, CD34, or a combination thereof and/or fibroblasts expressing markers such as but not limited to CD90, CD73, CD105 and HLA-G may be purified by positive or negative selection using techniques such as magnetic activated cell sorting (MACS), affinity columns, FACS, panning, other means known in the art, or a combination thereof. In some embodiments, cord blood-derived endothelial progenitor cells are administered directly into the target tissue, while in other embodiments, the cells are administered systemically. In some embodiments, the endothelial precursor cells are differentiated in vitro and infused into a patient. Verification of endothelial differentiation is performed by assessing ability of cells to bind FITC-labeled Ulex europaeus agglutinin-1, ability to endocytose acetylated Di-LDL, and the expression of endothelial cell markers such as PECAM-1, VEGFR-2, or CD31.

In some embodiments, cord blood fibroblast cells are endowed with desired activities prior to administration into the patient. In one specific embodiment, cord blood cells are “activated” ex vivo by brief culture in hypoxic conditions to upregulate nuclear translocation of the HIF-1α transcription factor and endow the cord blood cells with enhanced angiogenic potential. In some embodiments, hypoxia is achieved by culture of cells in conditions of 0.1% oxygen to 10% oxygen. In further embodiments, hypoxia is achieved by culture of cells in conditions of 0.5% oxygen and 5% oxygen. In further embodiments, hypoxia is achieved by culture of cells in conditions of about 1% oxygen. Cells may be cultured for a variety of time points ranging from 1 hour to 72 hours in some embodiments, to 13 hours to 59 hours in further embodiments and around 48 hours in still further embodiments. In one embodiment, cord blood cells are assessed for angiogenic or other desired activities prior to administration of the cord blood cells into the patient. Assessment methods are known in the art and include measurement of angiogenic factors, the ability to support cell viability and activity, and the ability to induce regeneration of the cellular components.

In additional embodiments, cord blood fibroblast cells are endowed with additional therapeutic properties through treatment ex vivo with factors such as de-differentiating compounds, proliferation-inducing compounds, compounds known to endow and/or enhance cord blood cells with useful properties, or a combination thereof. In one embodiment, cord blood cells are cultured with an inhibitor of the enzyme GSK-3 to enhance expansion of cells with pluripotent characteristics while maintaining the rate of differentiation. In another embodiment, cord blood cells are cultured in the presence of a DNA methyltransferase inhibitor such as 5-azacytidine to confer a “de-differentiation” effect. In another embodiment cord blood fibroblast cells are cultured in the presence of a differentiation agent that induces the cord blood stem cells to generate enhanced numbers of cells useful for treatment after the cord blood cells are administered to a patient.

In one embodiment, regenerative fibroblasts are purified from placental tissues. In contrast to cord blood fibroblast cells, in some embodiments, placental fibroblast cells are purified directly from placental tissues including the chorion, amnion, and villous stroma. In another embodiment, placental tissue is mechanically degraded in a sterile manner and treated with enzymes to allow dissociation of the cells from the extracellular matrix. Such enzymes include but are not restricted to trypsin, chymotrypsin, collagenases, elastase, hyaluronidase, or a combination thereof. In some embodiments, placental cell suspensions are subsequently washed, assessed for viability, and used directly by administration locally or systemically. In some embodiments, placental cell suspensions are purified to obtain certain populations with increased biological activity.

Purification may be performed using means known in the art including those used for purification of cord blood fibroblast cells. In some embodiments, purification may be achieved by positive selection for cell markers including SSEA3, SSEA4, TRA1-60, TRA1-81, c-kit, and Thy-1. In some embodiments, cells are expanded before introduction into the human body. Expansion can be performed by culture ex vivo with specific growth factors. Embodiments described for cord blood and embryonic stem cells also apply to placental stem cells.

Fibroblasts can also be extracted from various tissues including but not limited to umbilical cord, skin, adipose tissue, bone marrow, cord blood, and omental tissue. In some embodiments, fibroblasts are obtained from a source selected from the group comprising dermal fibroblasts; placental fibroblasts; omental tissue fibroblasts; adipose fibroblasts; bone marrow fibroblasts; foreskin fibroblasts; umbilical cord fibroblasts; cord blood fibroblasts; amniotic fluid; embryonic fibroblasts; hair follicle-derived fibroblasts; nail-derived fibroblasts; endometrial-derived fibroblasts; keloid-derived fibroblasts; ear lobe skin; plastic surgery-related by-products; or a combination thereof. In some embodiments, fibroblasts are fibroblasts isolated from skin, placenta, omentum, adipose tissue, bone marrow, foreskin, umbilical cord, cord blood, amnion, embryos, hair follicle, nails, endometrium, keloids, ear lobe, Wharton’s Jelly, and/or plastic surgery-related by-products.

In some embodiments, the fibroblasts are fibroblasts isolated from peripheral blood of a subject who has been exposed to conditions sufficient to stimulate fibroblasts from the subject to enter the peripheral blood. In another embodiment, fibroblast cells are mobilized by use of a mobilizing agent or therapy for treatment of ovarian failure. In some embodiments, the conditions and/or agents sufficient to stimulate fibroblasts from the subject to enter the peripheral blood comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof. In some embodiments, the mobilization therapy is selected from a group comprising exercise, hyperbaric oxygen, autohemotherapy by ex vivo ozonation of peripheral blood, induction of SDF-1 secretion in an anatomical area outside of the bone marrow, or a combination thereof. In some embodiments, the committed fibroblasts can express the marker CD133 or CD34 and are mobilized.

Any of the fibroblast cell populations disclosed herein may be used as a source of conditioned media and/or exosomes, apoptotic bodies, or microvesicles produced by fibroblasts. The cells may be cultured alone, or may by cultured in the presence of other cells in order to further upregulate production of growth factors in the conditioned media. In some embodiments, fibroblasts of the present disclosure are cultured with an inhibitor of mRNA degradation. In some embodiments, fibroblasts are cultured under conditions suitable to support differentiation and/or reprogramming of the fibroblasts. In some embodiments, such conditions comprise temperature conditions of between 30° C. and 38° C., between 31° C. and 37° C., or between 32° C. and 36° C. In some embodiments, such conditions comprise glucose at or below 4.6 g/l, 4.5 g/l, 4 g/l, 3 g/l, 2 g/l or 1 g/l. In some embodiments, such conditions comprise glucose of about 1 g/l.

Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time.” Fibroblast cells used in the disclosed methods can undergo at least 25, 30, 35, or 40 doublings prior to reaching a senescent state. Methods for deriving cells capable of doubling to reach 10¹⁴ cells or more are provided. In some embodiments, methods are used to derive cells that can double sufficiently to produce at least about 10¹⁴, 10¹⁵, 10¹⁶, or 10¹⁷ or more cells when seeded at from about 10³ to about 10⁶ cells/cm² in culture within 80, 70, or 60 days or less. In some embodiments, fibroblasts are transfected with one or more genes to allow for enhanced growth and overcoming of the Hayflick limit.

When referring to cultured cells, including fibroblast cells and vertebrae cells, the term senescence (also “replicative senescence” or “cellular senescence”) refers to a property attributable to finite cell cultures; namely, their inability to grow beyond a finite number of population doublings (sometimes referred to as Hayflick’s limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are reintroduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

In some embodiments, the present disclosure utilizes exosomes derived from fibroblasts as a therapeutic modality. Exosomes derived from fibroblasts may be used in addition to, or in place of, fibroblasts in the various methods and compositions disclosed herein. Exosomes, also referred to as “microparticles” or “particles,” may comprise vesicles or a flattened sphere limited by a lipid bilayer. The microparticles may comprise diameters of 40-100 nm. The microparticles may be formed by inward budding of the endosomal membrane. The microparticles may have a density of about 1.13-1.19 g/ml and may float on sucrose gradients. The microparticles may be enriched in cholesterol and sphingomyelin, and lipid raft markers such as GM1, GM3, flotillin and the src protein kinase Lyn. The microparticles may comprise one or more proteins present in fibroblast, such as a protein characteristic or specific to the fibroblasts or fibroblast conditioned media. They may comprise RNA, for example miRNA. The microparticles may possess one or more genes or gene products found in fibroblasts or medium which is conditioned by culture of fibroblasts. The microparticles may comprise molecules secreted by the fibroblasts. Such a microparticle, and combinations of any of the molecules comprised therein, including in particular proteins or polypeptides, may be used to supplement the activity of, or in place of, the fibroblasts for the purpose of, for example, treating or preventing ovarian failure. The microparticle may comprise a cytosolic protein found in cytoskeleton e.g., tubulin, actin and actin-binding proteins, intracellular membrane fusions and transport, e.g., annexins and rab proteins, signal transduction proteins, e.g., protein kinases, 14-3-3 and heterotrimeric G proteins, metabolic enzymes, e.g., peroxidases, pyruvate and lipid kinases, and enolase-1 and the family of tetraspanins, e.g., CD9, CD63, CD81 and CD82. In particular, the microparticle may comprise one or more tetraspanins.

As disclosed herein, fibroblasts may secrete one or more factors prior to or following introduction into an individual. Such factors include, but are not limited to, growth factors, trophic factors and cytokines. In some instances, the secreted factors can have a therapeutic effect in the individual. In some embodiments, a secreted factor activates the same cell. In some embodiments, the secreted factor activates neighboring and/or distal endogenous cells. In some embodiments, the secreted factor stimulated cell proliferation and/or cell differentiation. In some embodiments, fibroblasts secrete a cytokine or growth factor selected from human growth factor, fibroblast growth factor, nerve growth factor, insulin-like growth factors, hematopoietic stem cell growth factors, a member of the fibroblast growth factor family, a member of the platelet-derived growth factor family, a vascular or endothelial cell growth factor, and a member of the TGFβ family.

In some embodiments, fibroblasts are manipulated such that they do not produce one or more factors. Under appropriate conditions, fibroblasts may be capable of producing interleukin-1 (IL-1) and/or other inflammatory cytokines. In some embodiments, fibroblasts of the present disclosure are modified (e.g., by gene editing) to prevent or reduce expression of IL-1 or other inflammatory cytokines. For example, in some embodiments, fibroblasts are fibroblasts having a deleted or non-functional IL-1 gene, such that the fibroblasts are unable to express IL-1. Such modified fibroblasts may be useful in the therapeutic methods of the present disclosure by having limited pro-inflammatory capabilities when provided to a subject. In some embodiments, fibroblasts are treated with (e.g., cultured with) TNF-α, thereby inducing expression of growth factors and/or fibroblast proliferation.

In some embodiments, fibroblasts are transfected with one or more angiogenic genes to enhance ability to promote ovary function. An “angiogenic gene” describes a gene encoding for a protein or polypeptide capable of stimulating or enhancing angiogenesis in a culture system, tissue, or organism. Examples of angiogenic genes which may be useful in transfection of fibroblasts include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF. Fibroblasts transfected with one or more angiogenic factors may be used in the disclosed methods of disease treatment or prevention.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-2, FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and/or TGFβ-3. Factors from manipulated or stimulated fibroblasts may be present in conditioned media and collected for therapeutic use.

Conditions promoting certain types of fibroblast proliferation or differentiation can also be used during the culture of regenerative fibroblasts cells. These conditions include but are not limited to one or more of alteration in temperature, oxygen/carbon dioxide content, and/or turbidity of the growth media, and/or exposure to small molecule modifiers of cell culture like nutrients, certain enzyme inhibitors, certain enzyme stimulators, and/or histone deacetylase inhibitors, such as valproic acid.

In some embodiments, fibroblast cells are cultured under conditions to suppress expression of one or more apoptosis-associated genes. Anti-apoptosis genes allow for enhanced survival of fibroblasts in vitro and in vivo. In some embodiments, the one or more apoptosis-associated genes are selected from the group consisting of Fas, FasL, CASP1 (ICE), CASP10 (MCH4), CASP14, CASP2, CASP3, CASP4, CASP5, CASP6, CASP7, CASP8, CASP9, CFLAR (CASPER), CRADD, PYCARD (TMS1/ASC), ABL1, AKT1, BAD, BAK1, BAX, BCL2L11, BCLAF1, BID, BIK, BNIP3, BNIP3L, CASP1 (ICE), CASP10 (MCH4), CASP14, CASP2, CASP4, CASP6, CASP8, CD70 (TNFSF7), CIDEB, CRADD, FADD, FASLG (TNFSF6), HRK, LTA (TNFB), NOD1 (CARD4), PYCARD (TMS1/ASC), RIPK2, TNF, TNFRSF10A, TNFRSF10B (DR5), TNFRSF25 (DR3), TNFRSF9, TNFSF10 (TRAIL), TNFSF8, TP53, TP53BP2, TRADD, TRAF2, TRAF3, TRAF4, and a combination thereof.

In some embodiments, the conditions to suppress expression of apoptosis-associated genes comprise administration of an antisense oligonucleotide which activates RNAse H. In some embodiments, the conditions to suppress expression of apoptosis-associated genes comprise administration of an agent capable of inducing RNA interference, including short interfering RNA and/or short hairpin RNA.

In some embodiments, fibroblasts are cultured under hypoxic conditions prior to administration in order to confer enhanced cytokine production properties and stimulate migration toward chemotactic gradients. Without wishing to be bound theory, protocols to enhance the regenerative potential of non-fibroblast cells using hypoxia can be modified or adapted for use with fibroblasts. For example, in one study, short-term exposure of MSCs to 1% oxygen increased mRNA and protein expression of the chemokine receptors CX3CR1 and CXCR4. After 1-day exposure to low oxygen, in vitro migration of MSCs in response to the fractalkine and SDF-1alpha increased in a dose dependent manner, while blocking antibodies for the chemokine receptors significantly decreased migration. Xenotypic grafting of cells from hypoxic cultures into early chick embryos demonstrated more efficient grafting of cells from hypoxic cultures compared to cells from normoxic cultures, and cells from hypoxic cultures generated a variety of cell types in host tissues. Other descriptions of hypoxic conditioning are described in the art. For example, cells can be cultured in hypoxic conditions or with gases that displace oxygen and/or cells can be treated with hypoxic mimetics.

In some embodiments chemical agents such as iron chelators, for example, deferoxamine, are added during in vitro incubation or in vivo to enhance migration of fibroblasts to an area in need. Another useful preconditioning agent is all trans retinoic acid, used at concentrations similar to those described for MSC, for example, between 0.001 uM to 1 uM or between 0.01 µM and 1 µM.

Without wishing to be bound by theory, hypoxia has been demonstrated to induce expression of angiogenic genes in cells. For example, studies involving hypoxic preconditioning (HPC) of MSC exposed MSCs to 0.5% oxygen for 24, 48, or 72 h before evaluating the expression of prosurvival, proangiogenic, and functional markers, such as hypoxia-inducible factor-1α, VEGF, phosphorylated Akt, survivin, p21, cytochrome c, caspase-3, caspase-7, CXCR4, and c-Met. MSCs exposed to 24-h hypoxia showed reduced apoptosis and had significantly higher levels of prosurvival, proangiogenic, and prodifferentiation proteins compared to MSCs exposed to 72-h hypoxia. Cells taken directly from a cryopreserved state did not respond as effectively to 24-h HPC as those cells cultured under normoxia before HPC. Cells cultured under normoxia before HPC showed decreased apoptosis and enhanced expression of connexin-43, cardiac myosin heavy chain, and CD31. The preconditioned cells were also able to differentiate into cardiovascular lineages. The results of the study suggest that MSCs cultured under normoxia before 24-h HPC are in a state of optimal expression of prosurvival, proangiogenic, and functional proteins that may increase subsequent survival after engraftment of the cells. The same conditions used to culture MSCs can be used to culture the fibroblasts of the present disclosure.

Thus, in some embodiments, regenerative fibroblast cells are exposed to 0.1% to 10% oxygen for a period of 30 minutes to 3 days. In some embodiments, regenerative fibroblast cells are exposed to 3% oxygen for 24 hours. In some embodiments, regenerative fibroblast cells are exposed to cobalt (II) chloride to chemically induce hypoxia. In some embodiments, regenerative fibroblast cells are exposed to cobalt (II) chloride for 1 to 48 hours. In some embodiments, regenerative fibroblast cells are exposed to cobalt (II) chloride for 24 hours. In some embodiments, regenerative fibroblast cells are exposed to 1 µM-300 µM. In some embodiments, regenerative fibroblast cells are exposed to 250 µM cobalt (II) chloride. In some embodiments, hypoxia induces an upregulation in HIF-1α, which is detected by expression of VEGF secretion. In some embodiments, hypoxia induces an upregulation of CXCR4 on fibroblast cells, which promotes homing of the cells to an SDF-1 gradient in inflamed areas.

In some embodiments, the method optionally includes enriching populations of fibroblast cells. In one embodiment, cells are transfected with a polynucleotide vector containing a stem cell-specific promoter operably linked to a reporter or selection gene. In some embodiments, the cell-specific promoter is an Oct-4, Nanog, Sox-9, GDF3, Rex- 1, or Sox-2 promoter. In some embodiments, the method further includes the step of enriching the population for the regenerative fibroblast cells using expression of a reporter or selection gene. In some embodiments, the method further includes the step of enriching the population of the regenerative fibroblast cells by flow cytometry. In a further embodiment, the method further comprises the steps of selecting fibroblast cells expressing CD105 and/or CD 117 and transfecting the fibroblast cells expressing CD105 and/or CD 117 with cell-permanent NANOG gene.

In another embodiment, the method further includes the steps of contacting the fibroblast cells with a detectable compound that enters the cells, the compound being selectively detectable in proliferating and non-proliferating cells and enriching the population of cells for the proliferating cells. In some embodiments, the detectable compound is carboxyfluorescein diacetate, succinimidyl ester, or Aldefluor.

In some embodiments, fibroblast regenerative cells comprise fibroblast side population cells isolated based on expression of the multidrug resistance transport protein (ABCG2) or the ability to efflux intracellular dyes such as rhodamine-123 and or Hoechst 33342. Without being bound to theory, cells possessing this property express stem-like genes and are known for enhanced regenerative ability compared to cells which do not possess this efflux mechanism. Fibroblast side population cells are derived from tissues including pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer’s patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, or a combination thereof.

### II. Generation of Regenerative Fibroblast-Conditioned Media

Certain aspects of the present disclosure relate to methods of augmenting manipulation of the microbiome and/or treating and/or preventing dysbiosis through the therapeutic administration of concentrated media conditioned by regenerative fibroblast cells. In some embodiments, administration of fibroblast-conditioned culture media can induce the generation of CD8 cells. Methods for generating conditioned media from fibroblasts are described herein.

In some embodiments, regenerative fibroblast cells are cultured in a growth medium to obtain conditioned media. In some embodiments, fibroblasts are cultured directly in tissue culture media including DMEM, EMEM, IMEM, or RPMI to produce fibroblast-conditioned media. In some embodiments, fibroblast-conditioned media is generated by culturing fibroblasts in hypoxic and/or hyperthermic conditions and/or with histone deacetylase inhibitors. In some embodiments, regenerative fibroblasts are also cultured alone or cultured in the presence of other cells to further upregulate production of growth factors in the conditioned media. In some embodiments, cultured fibroblasts produce at least about 20 ng/ml FGF2, at least about 15 ng/ml FGF-2, at least about 10 ng/ml FGF-2, at least about 9 ng/ml FGF-2, at least about 8 ng/ml FGF-2, at least about 7 ng/ml FGF-2, at least about 6 ng/ml FGF-2, at least about 5 ng/ml FGF-2, at least about 4 ng/ml FGF-2, at least about 3 ng/ml FGF-2, at least about 2 ng/ml FGF-2, at least about 1 ng/ml FGF-2, at least about 0.5 ng/ml FGF-2, at least about 0.1 ng/ml FGF-2, at least about 0.05 ng/ml FGF-2, or at least about 0.01 ng/ml FGF-2. In some embodiments, cultured fibroblasts produce at least about 50 pg/ml TGF-β, at least about 45 pg/ml TGF-β, at least about 40 pg/ml TGF-β, at least about 35 pg/ml TGF-β, at least about 30 pg/ml TGF-β, at least about 29 pg/ml TGF-β, at least about 28 pg/ml TGF-β, at least about 27 pg/ml TGF-β, at least about 26 pg/ml TGF-β, at least about 25 pg/ml TGF-β, at least about 24 pg/ml TGF-β, at least about 23 pg/ml TGF-β, at least about 22 pg/ml TGF-β, at least about 21 pg/ml TGF-β, at least about 20 pg/ml TGF-β, at least about 19 pg/ml TGF-β, at least about 18 pg/ml TGF-β, at least about 17 pg/ml TGF-β, at least about 16 pg/ml TGF-β, at least about 15 pg/ml TGF-β, at least about 14 pg/ml TGF-β, at least about 13 pg/ml TGF-β, at least about 12 pg/ml TGF-β, at least about 11 pg/ml TGF-β, at least about 10 pg/ml TGF-β, at least about 5 pg/ml TGF-β, or at least about 1 pg/ml TGF-β.

Conditioned medium may be obtained from culture with fibroblasts. The cells may be cultured for about 1, 2, 3, 4, 5, 6, 7, 8, 9, 10 days or more. In some embodiments, the fibroblasts are cultured for about 3 days prior to collecting conditioned media. Conditioned media may be obtained by separating the cells from the media. Conditioned media may be centrifuged (e.g., at 500 × g). Conditioned media may be filtered through a membrane. The membrane may be a >1000 kDa membrane. Conditioned media may be subject to liquid chromatography such as HPLC. Conditioned media may be separated by size exclusion.

Fibroblasts may be expanded and utilized by administration themselves, or may be cultured in a growth media in order to obtain conditioned media. The term Growth Medium generally refers to a medium sufficient for the culturing of fibroblasts. In particular, one presently preferred medium for the culturing of the cells herein comprises Dulbecco’s Modified Essential Media (DMEM). Particularly preferred is DMEM-low glucose (also DMEM-LG herein) (Invitrogen®, Carlsbad, Calif.). The DMEM-low glucose is preferably supplemented with 15% (v/v) fetal bovine serum (e.g. defined fetal bovine serum, HycloneTM, Logan Utah), antibiotics/antimycotics (preferably penicillin (100 Units/milliliter), streptomycin (100 milligrams/milliliter), and amphotericin B (0.25 micrograms/milliliter), (Invitrogen®, Carlsbad, Calif.)), and 0.001% (v/v) 2-mercaptoethanol (Sigma®, St. Louis Mo.). In some cases different growth media are used, or different supplementations are provided, and these are normally indicated as supplementations to Growth Medium. Also relating to the present disclosure, the term standard growth conditions, as used herein refers to culturing of cells at 37° C., in a standard atmosphere comprising 5% CO₂, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

In one embodiment, the conditioned media comprises a liquid which has been in contact with fibroblast cells. In some embodiments, conditioned media is generated by culturing fibroblasts. In some embodiments, conditioned media is generated by combining fibroblasts with immune cells in a liquid media. Fibroblast cells may be fibroblasts that grow in an undifferentiated state and/or may be fibroblasts which have been stimulated with an agent that mimics inflammation and/or danger signals. Such agents are administered to fibroblasts in order to upregulate production of cytokines that are useful for the treatment of ovarian failure. In some embodiments cytokines secreted by fibroblasts whose upregulation is stimulated by exposure to inflammatory-related signals includes one or more of MCP-1, IL-6, IL-8, GCP-2, HGF, KGF, FGF, HB-EGF, BDNF, TPO, MIP1a, RANTES, TIMP1, HMGB1, IL-18, IL-33, or a combination thereof. In some embodiments fibroblasts are stimulated with agonists of toll-like receptors, including one or more of Triacyl lipopeptides, lipopeptides, lipoteichoic acid, HSP70, zymosan (Beta-glucan), double-stranded RNA, poly I:C, lipopolysaccharide, fibrinogen, heparan sulfate fragments, hyaluronic acid fragments, Bacterial flagellin, multiple diacyl lipopeptides, imidazoquinoline, loxoribine (a guanosine analogue), bropirimine, resiquimod, unmethylated CpG Oligodeoxynucleotide DNA, triacylated lipopeptides, Profilin, bacterial ribosomal RNA sequence “CGGAAAGACC,” or a combination thereof.

In one embodiment, provided is a means of creating a medicament useful for the treatment of dysbiosis by culturing cells in a serum free media. Many types of media may be chosen and used by one of skill in the art. In one embodiment, a media is selected from the group consisting of alpha MEM, DMEM, RPMI, Opti-MEM, IMEM, and AIM-V. Cells may be cultured in a variety of expansion media that contain fetal calf serum or other growth factors. However, for collection of therapeutic supernatant, in a specific embodiment, the cells are transferred to a media substantially lacking serum. In some embodiments, the supernatant is administered directly into the patient in need of treatment. It is well known in the art that preparation of the supernatant before administration may be performed by various means; for example, the supernatant may be filter sterilized or in some conditions concentrated. In a specific embodiment, the supernatant is administrated intramuscularly, intravenously, orally, sublingually, intranasally, intraventricularly, intrarectally, or intrathecally.

In some embodiments, culture conditioned media is concentrated by filtering/desalting means known in the art. In one embodiment, filters with specific molecular weight cut-offs are utilized. In one embodiments, the filters select for molecular weights between 1 kDa and 50 kDa. In one embodiment, the cell culture supernatant is concentrated using means known in the art such as solid phase extraction using C18 cartridges (Mini-Speed C18-14%, S.P.E. Limited, Concord ON). C18 cartridges are used to adsorb small hydrophobic molecules from the stem or progenitor cell culture supernatant, and allows for the elimination of salts and other polar contaminants. The cartridges are prepared by washing with methanol, followed by washing with deionized-distilled water. In some embodiments, up to 100 ml of stem cell or progenitor cell supernatant may be passed through each of these specific cartridges before elution, though one of skill in the art would understand that larger cartridges may be used. After washing the cartridges, adsorbed material is eluted with methanol, evaporated under a stream of nitrogen, re-dissolved in a small volume of methanol, and stored at 4° C. Before testing the eluate for activity in vitro, the methanol is evaporated under nitrogen and replaced by culture medium. In other embodiments, different adsorption means known in the art are used to purify certain compounds from fibroblast cell supernatants.

In some embodiments, further purification and concentration is performed using gel filtration with a Bio-Gel P-2 column having a nominal exclusion limit of 1800 Da (Bio-Rad, Richmond Calif.). The column is washed and pre-swelled in 20 mM Tris-HCl buffer, pH 7.2, (Sigma) and degassed by gentle swirling under vacuum. Bio-Gel P-2 material is packed into a 1.5×0.54 cm glass column and equilibrated with 3 column volumes of the same buffer. Cell supernatant concentrates extracted by filtration are dissolved in 0.5 ml of 20 mM Tris buffer, pH 7.2, and run through the column. Fractions are collected from the column and analyzed for biological activity. In alternative embodiments, other purification, fractionation, and identification means known to one skilled in the art including anionic exchange chromatography, gas chromatography, high performance liquid chromatography, nuclear magnetic resonance, and mass spectrometry are used to prepare concentrated supernatants.

In some embodiments, active supernatant fractions are administered locally or systemically. The supernatant concentrated from fibroblast-conditioned media is assessed directly for biological activities or further purified. In vitro bioassays allow for identification of the molecular weight fraction of the supernatant possessing biological activity and quantification of biological activity within the identified fractions. Production of various proteins and biomarkers associated is assessed by analysis of protein content using techniques including mass spectrometry, column chromatography, immune based assays such as enzyme linked immunosorbent assay (ELISA), immunohistochemistry, and flow cytometry.

### III. Exosomes Purified From Fibroblasts

In some embodiments, exosomes purified from transfected fibroblasts are used therapeutically. In some embodiments, exosomes purified from fibroblast-conditioned media are used therapeutically. In some embodiments, purified fibroblast exosomes are used to regenerate functional tissue, induce proliferation, augment manipulation of the microbiome, and/or treat and/or prevent dysbiosis. In some embodiments, fibroblast-derived exosomes are used to suppress inflammation, including but not limited to suppressing production of IL-1, IL-6, and TNF-α by macrophages. Methods for purifying exosomes are known in the art and described herein. The exosomes can express CD81 and/or membrane-bound TNG-β. Further, the exosomes can be at least about 10-1000 nm, at least about 25-800 nm, at least about 40-600 nm, at least about 50-400 nm, or at least about 60-200 nm in size.

In one embodiment, fibroblasts are cultured using means known in the art for preserving the viability and proliferative ability of fibroblasts. Both individualized autologous exosome preparations and exosome preparations obtained from established cell lines for experimental or biological use may be used. In one embodiment, chromatography separation methods are used to prepare membrane vesicles, particularly to separate the membrane vesicles from potential biological contaminants, wherein the membrane vesicles are exosomes and cells used to generate the exosomes are fibroblast cells.

In one embodiment, strong or weak anion exchange is performed. In addition, in a specific embodiment, the chromatography is performed under pressure. Thus, in some embodiments, the chromatography consists of high performance liquid chromatography (HPLC). Different types of column supports may be used to perform the anion exchange chromatography. In some embodiments, the column supports include cellulose, poly(styrenedivinylbenzene), agarose, dextran, acrylamide, silica, ethylene glycol-methacrylate co-polymer, or mixtures thereof, e.g., agarose-dextran mixtures. Column supports include but are not limited to gels including: SOURCE™, POROS™, SEPHAROSE™, SEPHADEX™, TRISACRYL™, TSK-GEL SW™ or PW™, SUPERDEX™, TOYOPEARL HW™, and SEPHACRYL™. Therefore, in a specific embodiment, membrane vesicles, particularly exosomes, are prepared from a biological sample such as a tissue culture containing fibroblasts, comprising at least one step during which the biological sample is purified by anion exchange chromatography on an optionally-functionalized column support selected from one or more of cellulose, poly(styrene-divinylbenzene), silica, acrylamide, agarose, dextran, ethylene glycol-methacrylate co-polymer, alone or in combinations thereof.

In some embodiments, the column supports are in bead form to improve chromatographic resolution. The beads can be homogeneous and calibrated in diameter with a sufficiently high porosity to enable the penetration of objects like exosomes undergoing chromatography. The diameter of exosomes is generally between 50 and 100 nm. Thus, in some embodiments, high porosity gels with diameters between about 10 nm and about 5 µm, about 20 nm and about 2 µm, or about 100 nm and about 1 µm are used. For anion exchange chromatography, the column support used can be functionalized with a group capable of interacting with an anionic molecule. Generally, this group is composed of a ternary or quaternary amine, which defines a weak or strong anion exchanger, respectively. In some embodiments, a strong anion exchanger corresponding to a chromatography column support functionalized with quaternary amines is used. Therefore, according to a more specific embodiment, anion exchange chromatography is performed on a column support functionalized with a quaternary amine and selected from one or more of poly(styrenedivinylbenzene), acrylamide, agarose, dextran, and silica, alone or in combinations thereof, and functionalized with a quaternary amine. Column supports functionalized with a quaternary amine include but are not limited to gels including SOURCE™ Q, MONO Q™, Q SEPHAROSE™, POROS™ HQ and POROS™ QE, FRACTOGEL™ TMAE type gels and TOYOPEARL SUPER™ Q gels.

In one embodiment, the column support used to perform the anion exchange chromatography comprises poly(styrene-divinylbenzene), for example, SOURCE Q gels like SOURCE™ 15Q (Pharmacia). This column support comprises large internal pores, low resistance to liquid circulation through the gel, and rapid diffusion of exosomes to the functional groups. Biological materials including exosomes retained on the column support may be eluted using methods known in the art, for example, by passing a saline solution gradient of increasing concentration over the column support. In some embodiments, a sodium chloride solution is used in concentrations varying from 0 to 2 M, for example. Purified fractions are detected based on optical densities (OD) of the fractions measured at the column support outlet using a continuous spectrophotometric reading. In some embodiments, fractions comprising membrane vesicles are eluted at an ionic strength of approximately 350 to 700 mM, depending on vesicle type.

Different types of chromatographic columns may be used depending on experimental requirements and volumes to be purified. For example, depending on the preparations, column volumes can vary from 100 µl up to ≥10 ml, and column supports can bind and retain up to 25 mg of proteins/ml. As an example, a 100 µl column has a capacity of approximately 2.5 mg of protein, which allows for purification of approximately 2 liters of culture supernatants concentrated by a factor of 10 to 20 to yield volumes of 100 to 200 ml per preparation. Higher volumes may also be purified by increasing the column volume.

Membrane vesicles can also be purified using gel permeation liquid chromatography. In some embodiments, the anion exchange chromatography step is combined with a gel permeation chromatography step either before or after the anion exchange chromatography step. In some embodiments, the permeation chromatography step takes place after the anion exchange step. In some embodiments, the anion exchange chromatography step is replaced with the gel permeation chromatography step. To perform gel permeation chromatography, a support selected from one or more of silica, acrylamide, agarose, dextran, ethylene glycol-methacrylate co-polymer, alone or in combinations thereof, e.g., agarose-dextran mixtures. Column supports include but are not limited to gels including SUPERDEX™ 200HR (Pharmacia), TSK G6000 (TosoHaas), and SEPHACRYL™ S (Pharmacia).

Gel permeation chromatography may be applied to different biological samples. In some embodiments, biological samples include but are not limited to biological fluid from a subject (bone marrow, peripheral blood, etc.), cell culture supernatant, cell lysate, pre-purified solution, or any other composition comprising membrane vesicles. In one embodiment, the biological sample is a culture supernatant of membrane vesicle-producing fibroblast cells treated, prior to chromatography, so as to enrich the supernatant for membrane vesicles. Thus, one embodiment relates to a method of preparing membrane vesicles from a biological sample, the method characterized by at least a) an enrichment step to prepare a sample enriched with membrane vesicles and b) a purification step during which the sample is purified by anion exchange chromatography and/or gel permeation chromatography.

In some embodiments, the biological sample is composed of a pre-purified solution enriched with membrane vesicles obtained by centrifugation, clarification, ultrafiltration, nanofiltration and/or affinity chromatography of a cell culture supernatant of a membrane vesicle-producing fibroblast cell population or biological fluid. Thus, one embodiment relates to a method of preparing membrane vesicles comprising at least the steps of a) culturing a population of membrane vesicle-producing cells under conditions enabling the release of vesicles; b) enriching membrane vesicles in the sample; and c) performing anion exchange chromatography and/or gel permeation chromatography to purify the sample.

In some embodiments, the sample (e.g. supernatant) enrichment step comprises one or more centrifugation, clarification, ultrafiltration, nanofiltration, affinity chromatography, or a combination thereof. In one embodiment, the enrichment step comprises the steps of (i) elimination of cells and/or cell debris (clarification) and (ii) concentration and/or affinity chromatography. In one embodiment, affinity chromatography following clarification is optional. In one embodiment, the enrichment step comprises the steps of (i) elimination of cells and/or cell debris (clarification); (ii) concentration; and (iii) an affinity chromatography.

In some embodiments, the elimination step of enrichment is achieved by centrifugation of the sample, for example, at a speeds below 1000 g, such as between 100 and 700 g. In one embodiment, centrifugation conditions are approximately 300 g or 600 g for a period between 1 and 15 minutes.

In some embodiments, the elimination step of enrichment is achieved by filtration of the sample. In some embodiments, sample filtration is combined with centrifugation as described. The filtration may be performed with successive filtrations using filters with a decreasing porosity. In one embodiment, filters with a porosity between 0.2 and 10 µm are used. In one embodiment, a succession of filters with a porosities of 10 µm, 1 µm, 0.5 µm, and 0.22 µm are used.

In some embodiments, the concentration step of enrichment is performed to reduce the volume of sample to be purified during chromatography. In some embodiments, the concentration step of enrichment is achieved by centrifugation of the sample at speeds between 10,000 and 100,000 g to cause the sedimentation of the membrane vesicles. In some embodiments, the concentration step of enrichment is performed as a series of differential centrifugations, with the last centrifugation performed at approximately 70,000 g. After centrifugation, the pelleted membrane may be resuspended in a smaller volume of suitable buffer.

In some embodiments, the concentration step of enrichment is achieved by ultrafiltration which allows both to concentration of the supernatant and initial purification of the vesicles. In one embodiment, the biological sample (e.g., the supernatant) is subjected to tangential ultrafiltration consisting of concentration and fractionation of the sample between two compartments (filtrate and retentate) separated by membranes of determined cut-off thresholds. Separation of the sample is carried out by applying a flow in the retentate compartment and a transmembrane pressure between the retentate compartment and the filtrate compartment. Different systems may be used to perform the ultrafiltration, such as spiral membranes (Millipore, Amicon), flat membranes, or hollow fibers (Amicon, Millipore, Sartorius, Pall, GF, Sepracor). In some embodiments, membranes with cut-off thresholds below 1000 kDa, 300 kDa to 1000 kDa, or 300 kDa to 500 kDa are used.

The affinity chromatography step can be performed in various ways, using different chromatographic support and material known in the art. In some embodiments, nonspecific affinity chromatography aimed at retaining (i.e., binding) certain contaminants present within the solution without retaining the objects of interest (i.e., the exosomes) is used as a form of negative selection. In some embodiments, affinity chromatography on a dye is used, allowing for the elimination (i.e., the retention) of contaminants such as proteins and enzymes like albumin, kinases, dehydrogenases, clotting factors, interferons, lipoproteins, or also co-factors, etc. The supports used for affinity chromatography on a dye are the supports used for ion exchange chromatography functionalized with a dye. In some embodiments, the dye is selected from the group consisting of Blue SEPHAROSE™ (Pharmacia), YELLOW 86, GREEN 5, and BROWN 10 (Sigma). In some embodiments, the support is agarose. However, those of skill in the art will understand that any other support and/or dye or reactive group allowing the retention (binding) of contaminants from the biological sample to be purified can be used.

Thus, one embodiment relates to a method of preparing membrane vesicles comprising the steps of a) culturing a population of membrane vesicle-producing cells under conditions enabling release of the vesicles; b) treating the culture supernatant with at least one ultrafiltration or affinity chromatography step to produce a biological sample enriched with membrane vesicles; and c) using anion exchange chromatography and/or gel permeation chromatography to purify the biological sample. In some embodiments, step b) comprises filtration of the culture supernatant, followed by an ultrafiltration, including tangential ultrafiltration. In further embodiments, step b) comprises clarification of the culture supernatant, followed by an affinity chromatography on dye, including Blue SEPHAROSE™.

In some embodiments, after step c), the harvested material is subjected to d) one or more additional treatment and/or filtration steps for sterilization purposes. For step d), filters with a diameter ≤0.3 µm or ≤0.25 µm are used. In some embodiments, after step d), the sterilized, purified material obtained is distributed into suitable containers such as bottles, tubes, bags, syringes, etc., in a suitable storage medium and stored cold, frozen, or used extemporaneously. Thus, in some embodiments, the method of preparing membrane vesicles further comprises c) anion exchange chromatography purification of the biological sample and d) a sterilizing filtration step of the material harvested in step c). In further embodiments, the method of preparing membrane vesicles further comprises c) gel permeation chromatography purification of the biological sample and d) a sterilizing filtration step of the material harvested in step c). In additional embodiments, the method of preparing membrane vesicles further comprises c) anionic exchange purification of the biological sample followed or preceded by gel permeation chromatography and d) a sterilizing filtration step of the material harvested in step c).

### IV. Methods and Compositions to Treat Dysbiosis

In some embodiments of the disclosure, the microbiome of a host is manipulated to counteract dysbiosis and optimize the host’s microbiome. Diet exerts major effects on the gut microbiota and is one of the main drivers in shaping the gut microbiome over time. The consumption of food or dietary supplements containing prebiotics or dietary fiber provides fuel for the bacteria residing in the gut and has been shown to reduce symptoms associated with various GI diseases and disorders. Thus, in some embodiments, diet can be altered to restore the gut microbiome.

Dysbiosis, including abnormalities in the microbiome of a host, can result from a genetic predisposition to dysbiosis and/or be induced by a treatment, for example. Treatments which can induce dysbiosis include but are not limited to antibiotics, chemotherapy, radiation therapy, immunotherapy, hormonal therapy, or a combination thereof. Assessment of dysbiosis can occur via ribosomal sequencing. The host microbiome can include commensal bacteria in the gut, blood, skin, epithelial tissue, bone marrow, thymus, spleen, liver, or a combination thereof.

In some embodiments, fecal microbiota transplantation can be used to restore the gut microbiome by supplying microbiome components from an external source. Fecal microbiota transplantation has also emerged as a potentially beneficial method for manipulating the gut microbiome. In this procedure, stool from a healthy donor is administered to an unhealthy subject. Changes in the microbiome of recipients have been observed and are likely due to the presence of healthy microbiota in the transplanted stool from healthy patients colonizing the unhealthy gut and re-establishing normal GI function and metabolic niches. While this therapy has been successful in the clinic, it has not been widely accepted because of concerns over infection transmission, lack of aesthetic appeal, and lack of knowledge of long-term effects within the recipient microbiome.

In some embodiments, pre- and/or probiotics can be administered to restore the gut microbiome. Administration of probiotics is a widely accepted method for restoring the gut microbiome. Probiotics are isolated colonies of live microorganisms that confer benefits to the host. These microorganisms are usually made up of commensal bacteria encapsulated for daily use. While there is growing evidence to support the use of probiotics for gastrointestinal health, there are limitations in the field of probiotics. Currently, many probiotics are composed of the same few commensal bacteria of the families Lactobacillus and Bifidobacterium. Additionally, the number of colony-forming units and the specific delivery system to achieve efficient colonization by the commensal bacteria are still relatively unknown. Therefore, there is a need for the discovery of new probiotic compositions to combat gastrointestinal dysbiosis.

In some situations, alterations in the human microbiome are characterized by a reduction in the diversity of bacterial families and disequilibrium in various groups of microbiota. Anaerobes in the colon ferment polysaccharides, which results in the production of short chain fatty acids (SCFA). The three main SCFAs are proprionate, acetate, and butyrate. SCFAs are the major energy source for cells in the intestines, contributing 60-70% of the energy derived from carbohydrates. SCFAs also contribute to host health through epithelial proliferation and differentiation. Butyrate in particular is considered to have an important role in regulation of digestive health.

Depletion of specific bacterial families such as Lachnospiracea and other butyric acid-producing bacteria are common in GI disorders. The depletion of butyric-acid producing bacteria is implicated in the development of colonic inflammation that is characteristic of C. difficile colitis, inflammatory bowel disease (IBD), irritable bowel disorder (IBS), and general antibiotic-associated microbial dysbiosis. The importance of butyrate in the GI tract has been known for some time, but the connection between the gut microbiome and this crucial SCFA was not made until recently. Colonic epithelial cells use butyrate as one of their main sources of energy. In addition to its role in fueling colonic cells, butyrate is also key in regulating cellular proliferation and differentiation. It has also been shown to produce anti-inflammatory effects by inhibiting the activation of transcription factor NF-KB, which leads to a reduction in pro-inflammatory cytokines. Thus, in some embodiments, butyrate supplements can be effective in ameliorating the inflammation caused by various GI diseases and disorders, including C. difficile colitis, Crohn’s disease, and IBS.

In some embodiments, treatment of the host with probiotics is performed to optimize the microbiome. Probiotics of the present disclosure, alone or in combination with one or more additional pre- and/or probiotics, include but are not limited to Bifidobacterium spp., Lactobacillus spp., Streptococcus thermophilia, Bacillus coagulans, Bacillus laterosporus, Pediococcus acidilactici, Saccharomyces boulardii, or a combination thereof. Treatment with one or more of these probiotics can affect the population in the gut of one or more bacterial species including but not limited to: Alistepes putredinis, Alistepes finegoldii, Anaerotruncus colihominis, Anaerofustis stercorihominis, Bacteroides thetaiotaomicron, Bacteroides vulgatus, Bacteroides ovatus, Bacteroides distasonis, Bacteroides salyersiae, Bacteroides stercoris, Bacteroides eggerthii, Bacteroides merdae, Bacteroides caccae, Bacteroides stercosis, Bacteroides uniformis, Bacteroides WH302, Bulleidia moorei, Bacteroides capillosus, Bifidobacterium animalis, Bifidobacterium breve, Bifidobacterium bifidum, Bifidobacterium longum, Bifidobacterium infantis, Bacillus coagulans, Clostridium leptum, Clostridium boltaea, Clostridium symbiosum, Clostridium scindens, Clostridium bartlettii, Clostridium spiroforme, Coprococcus catus, Catenibacterium mitsuokai, Coprococcus eutactus, Dorea formicigenerans, Dorea longicatena, Dialister sp., Eubacterium ventriosum, Eubacterium halii, Eubacterium siraeum, Eubacterium dolichum, Eubacterium cylindroides, Eubacterium biforme, Eubacterium plautii, Faecalibacterium prausnitzii, Gemella haemolysans, Lactobacilllus lactis, Lactobacillus acidophilus,Lactobacillus bulgaricus, Lactobacillus casei, Lactobacillus fermentum, Lactobacillus gasseri, Lactobacillus plantarum, Lactobacillus reuteri, Lactobacillus rhamnosus (e.g., GG), Lactobacillus paracasei, Lactobacillus plantarus, Lactobacillus rhamnosus, Lactobacillus reuteri, Lactobacillus salivarius, Peptostreptococcus micros, Ruminococcus gnavus, Ruminococcus obeum, Ruminococcus torques, Ruminococcus callidus, Roseburia faecalis, Ruminococcus bromii, Subdoligranulum variabile, Saccharomyces boulardii, Streptococcus thermophiles, Streptoccocus salivarius K12, or Streptoccocus Salivarius M18.

In some embodiments, treatment of the host with prebiotics is performed to optimize the microbiome. Prebiotics of the present disclosure, alone or in combination with one or more additional pre and/or probiotics, include but are not limited to soluble starch, yeast extract, polydextrose, polydextrose powder, lactulose, lactosucrose, raffinose, gluco-oligosaccharide, inulin, fructo-oligosaccharide, isomalto-oligosaccharide, soybean oligosaccharides, lactosucrose, xylo-oligosaccharide, chito-oligosaccharide, manno-oligosaccharide, aribino-oligosaccharide, siallyl-oligosaccharide, fuco-oligosaccharide, galacto-oligosaccharide, gentio-oligosaccharides, or a combination thereof.

In some embodiments, the one or more pre- and/or probiotics can alter the ratio to one another of various combinations of microbiota species in the gut of the subject, such as at least two species, at least three species, at least four species, at least five species, at least six species, at least seven species, at least eight species, at least nine species, or at least ten species. In some embodiments, the probiotic/enzyme mix can increase the presence of one or more bacterial, viral, or eukaryotic species that reside in the gut. In some embodiments, the composition can increase the presence of microbiota species that reside in the gut.

### V. Improving Therapeutic Efficacy of Regenerative Fibroblasts and Products Thereof by Treating Dysbiosis

In some embodiments of the disclosure, treatment of dysbiosis is performed so as to augment the innate regenerative ability of the host. In some embodiments, the regenerative abilities of fibroblasts administered to the host are augmented by treatment of dysbiosis prior to, at the same time as, and/or after administration of regenerative fibroblasts and/or regenerative-fibroblast conditioned media. In some embodiments, regenerative fibroblasts are used as an alternative to MSCs and other stem cell types. In some embodiments, the microbiome is repaired as a means of enhancing regenerative activity of fibroblasts. Methods of the disclosure also encompass increasing the efficacy of a fibroblast-based treatment comprising the steps of: a) treating dysbiosis in an individual in need thereof; and b) administering the fibroblast-based treatment, wherein treating the dysbiosis can occur prior to, at the same time as, and/or after administering the fibroblast-based treatment. The methods may further comprisethe steps of: identifying the individual in need thereof; and assessing dysbiosis in said individual.

Recent studies have focused on the use of adult stem cells for disorders such as degenerative disc disease. Mesenchymal stem cells (MSCs) are non-hematopoietic, multipotent progenitor cells, which can be isolated from various human adult tissues [30]. In recent years, MSCs have been shown to possess a broad range of regenerative capabilities, including modulating disease progression by repairing lesions closely associated with degenerative disc disease as a result of the ability of MSCs to form cells of multiple lineages [31-33].

Although MSCs possess various regenerative properties, issues with MSC extraction, expansion, and maintenance costs have a limiting effect on their widespread utilization. One potential substitute for MSCs are fibroblasts, which can be readily acquired in large numbers, are relatively easy to expand in vitro, and are economical to produce. Studies show fibroblasts possess a differentiation potential similar to MSCs. In one report, mechanical stimulation was applied to dermal fibroblast cells encapsulated in alginate beads using a custom-built bioreactor system for either a 1- or 3-week period at a frequency of 1 Hz for 4 h/day under hypoxic conditions. Chondrogenic differentiation of the fibroblasts was observed, as indicated by elevated aggrecan gene expression and an increased collagen production rate [34]. In vivo ability of fibroblasts to differentiate into chondrogenic cells was demonstrated in a subsequent study.

In another recent study, researchers induced disc degeneration in New Zealand white rabbits by annular puncture, and after 4 weeks, intradiscally implanted human dermal fibroblasts or saline. Eight weeks after cellular implantation, disc height increased significantly in the treated discs compared to saline controls. Further, cells in treated discs displayed reduced expression of inflammatory markers, a higher ratio of collagen type II over collagen type I gene expression, and more intense immunohistochemical staining for both collagen types I and II [35]. A subsequent study conducted by an independent group involved disc puncture in 8 rabbits to induce disc degeneration. One month later, cultured fibroblasts taken from the skin were injected into the disc. The viability and potential of the injected cells for reproduction were studied histologically and radiologically. The fibroblasts injected to the degenerated discs were viable [36]. Cellular formations and organizations indicative of histological recovery were observed in the discs to which fibroblasts were transplanted. The histological findings of the discs not transplanted with fibroblasts showed no histological recovery. Radiologically, no finding of the improvement was found in either groups.

In some embodiments, a probiotic/enzyme mixture is used repair dysbiosis and treat disc degeneration in combination with administration of regenerative fibroblasts. Disc degeneration is an inflammatory process [179]. Probiotics inhibit chronic inflammation [173-175]. There is some evidence that degenerative disc disease (DDD) is associated with Propionibacterium acnes infection inside the disc [180-182]. There is evidence that probiotics can suppress growth of Propionibacterium acnes [183].

In addition to differentiation into chondrocytic tissues, other studies have shown that fibroblasts are capable of differentiating into other types of cells. In one study, cultured human adult bronchial fibroblast-like cells (Br) where assessed in comparison with mesenchymal cell progenitors isolated from fetal lung (ICIG7) and adult bone marrow (BM212) tissues. Surface immunophenotyping by flow cytometry revealed a similar expression pattern of antigens characteristic of marrow-derived MSCs, including CD34 (-), CD45 (-), CD90/Thy-1 (+), CD73/SH3, SH4 (+), CD105/SH2 (+) and CD166/ALCAM (+) in Br, ICIG7, and BM212 cells. There was one exception, STRO-1 antigen, which was only weakly expressed in Br cells. Analysis of cytoskeleton and matrix composition by immunostaining showed that lung and marrow-derived cells homogeneously expressed vimentin and nestin proteins in intermediate filaments but were devoid of epithelial cytokeratins. Additionally, α-smooth muscle actin was also present in the microfilaments of a low number of cells. All cell types predominantly produced collagen and fibronectin extracellular matrix as evidenced by staining with the monoclonal antibodies for collagen prolyl 4-hydroxylase and fibronectin isoforms containing the extradomain (ED)-A together with ED-B in ICIG7 cells. Similar to fetal lung and marrow fibroblasts, Br cells were able to differentiate along the three adipogenic, osteogenic, and chondrogenic mesenchymal pathways when cultured under appropriate inducible conditions. Altogether, this data indicates that MSCs are present in human adult lung. They may be actively involved in lung tissue repair under physiological and pathological circumstances [37].

Further support for the ability of fibroblasts to differentiate into multiple lineages of cells comes from studies using cells isolated from juvenile foreskins. These cells where shown to share a mesenchymal stem cell phenotype and multi-lineage differentiation potential. Specifically, the investigators demonstrated similar expression patterns for CD14(-), CD29(+), CD31(-), CD34(-), CD44(+), CD45(-), CD71(+), CD73/SH3-SH4(+), CD90/Thy-1(+), CD105/SH2(+), CD133(-) and CD166/ALCAM(+) in well-established adipose tissue derived-stem cells and foreskin fibroblastic cells by flow cytometry. Immunostainings showed fibroblast cells expressed vimentin, fibronectin and collagen; they were less positive for α-smooth muscle actin and nestin, while they were negative for epithelial cytokeratins. When cultured under appropriate inducible conditions, both cell types could differentiate along the adipogenic and osteogenic lineages. Additionally, fibroblasts demonstrated a higher proliferation potential than mesenchymal stem cells. These findings are of particular importance, because skin or adipose tissues are easily accessible for cell transplantations in regenerative medicine [38]. Verification of multi-lineage differentiation of foreskin fibroblasts was provided by a study in which foreskin fibroblasts where demonstrated to possess shorter doubling time than MSCs, as well as ability to multiply more than 50 doublings without undergoing senescence. The cells were positive for the MSC markers CD90, CD105, CD166, CD73, SH3, and SH4, and could be induced to differentiate into osteocytes, adipocytes, neural cells, smooth muscle cells, Schwann-like cells, and hepatocyte-like cells [39].

One embodiment of the present disclosure encompasses the administration of a probiotic/enzyme mixture as a means of stimulating post-stroke angiogenesis. It has been previously reported that increased microvessel density in the peri-infarct area correlates with longer survival times in ischemic stroke patients [1-3]. This raises the possibility that enhancement of angiogenesis could be a useful strategy for facilitating functional recovery after ischemic stroke [4-7]. Intriguingly, blood vessels and axons are tightly joined and run parallel throughout the central nervous system, suggesting a coupling of blood vessel and axon components. It is known that conditions such as stroke cause ischemia, and ischemia stimulates production of new blood vessels through multiple mechanisms, including stimulation of hypoxia inducible factor (HIF)-1 activation and its nuclear translocation [8-13]. This results in increased transcription of numerous genes, including angiogenic genes such as SDF-1 [14-18] which attracts endothelial progenitor cells, genes such as VEGF [19, 20] which triggers formation of new blood vessels, and genes such as PD-L1 which suppresses inflammation in an attempt to restore new tissue growth without fibrosis. Other beneficial activities of HIF-1α after stroke include activation of endogenous stem cells and/or repair of endothelium inside the brain of the stroke victim [21-24] and protection of astrocytes from glutamate toxicity [25].

Post-ischemic angiogenesis may modulate 1) axonal outgrowth and 2) neurogenesis, including proliferation, migration, and maturation of neural stem and/or progenitor cells (NSCs), and post-ischemic angiogenesis is thought to contribute to functional recovery after stroke. Following ischemic stroke, administration of bone marrow cells may be correlated with improved regional cerebral blood flow, regional metabolic rate of oxygen consumption, and improved neurological function [26-77].

The therapeutic effects after stroke of other pro-angiogenic growth factors have also been described. For example, an FGF2-apatite coating was developed as a slow-releasing drug delivery system (DDS) by forming an FGF2/calcium phosphate composite layer. Hydroxyapatite was coated with high or low doses of FGF2, denoted as FGF-high and FGF-low. This study investigated the efficacy of the coating as an angiogenesis therapy for brain infarction. Rats were subjected to permanent occlusion of the middle cerebral artery, an FGF2- apatite-coated implant was inserted, and the rat brains were removed 2 weeks after implantation. Rats in groups treated with FGF-high had significantly smaller areas of brain infarction, particularly in the external capsule and the lateral side of the putamen, and better capillary density than rats in groups treated with non-FGF2-apatite-coated implants. Histologic analysis indicated that the new vessels were larger and had thicker walls in the FGF2-apatite-coated groups than in the non-FGF2 groups. Fluorescence immunohistochemistry of the peri-infarction region showed that FGF2 released from FGF2-apatite-coated implants might have biological activity. Moreover, fluorescence immunohistochemistry showed that released FGF2 influenced microglia cells [78].

In one interesting experiment, permanent middle cerebral artery occlusion was performed in mice whose bone marrow (BM) had been replaced with BM cells from green fluorescent protein (GFP)-transgenic mice. The occluded mice were treated with cytokines including granulocyte colony stimulating factor (G-CSF) and stem cell factor (SCF) in the acute phase (days 1 to 10) or subacute phase (days 11 to 20), and brain function and histological changes were evaluated. Separately, the researchers injected bromodeoxyuridine during cytokine treatment to assess cell kinetics in the brain. Six mice were prepared for each experimental group. Administration of G-CSF and SCF in the subacute phase effectively improved not only motor performance but also higher brain function compared with acute-phase treatment. Acute-phase and subacute-phase treatments identically reduced the infarct volume relative to vehicle treatment. However, subacute-phase treatment significantly induced transition of BM-derived neuronal cells into the peri-infarct area and stimulated proliferation of intrinsic neural stem/progenitor cells in the neuroproliferative zone. It was concluded that administration of G-CSF and SCF in the subacute phase after focal cerebral ischemia is effective for functional recovery, enhancing cytokine-induced generation of neuronal cells from both BM-derived cells and intrinsic neural stem/progenitor cells [79].

In another study, BM stem cells (BMSCs) were harvested from green fluorescent protein-transgenic mice and were cultured. The mice were subjected to permanent middle cerebral artery occlusion. BM stem cells or vehicle were transplanted into the ipsilateral striatum 7 days after the insult. Using autoradiography and fluorescence immunohistochemistry, the binding of 125I-iomazenil and the expression of GABA receptor protein in and around the cerebral infarct were evaluated 4 weeks after transplantation. It was found that binding of 125I-iomazenil was significantly higher in the peri-infarct neocortex in the BMSC-transplanted animals than in the vehicle-transplanted animals. Likewise, the number of the GABAA receptor-positive cells was significantly higher in the peri-infarct neocortex in the BMSC-transplanted animals than in the vehicle-transplanted animals. A certain subpopulation of the transplanted BMSCs expressed a neuron-specific marker, microtubule-associated protein 2, and the marker protein specific for GABAA receptor in the peri-infarct area. These findings suggest that BMSCs may contribute to neural tissue regeneration through migrating toward the peri-infarct area and acquiring the neuron-specific receptor function [80].

It does appear that in some studies, administered stem cells stimulate proliferation of endogenous neural progenitors, or at least contribute to protection of these cells, in part through stimulation of angiogenesis. For example, in one study, bone marrow-derived MSCs were transplanted into the brain parenchyma 3 days after induction of stroke by occluding the middle cerebral artery for 2 hours. Stroke induced proliferation of resident neural stem cells in the subventricular zone. However, most of nascent cells underwent cell death and had a limited impact on functional recovery after stroke. Transplantation of MSCs enhanced proliferation of endogenous neural stem cells while suppressing death of newly generated cells. Nascent cells migrated toward ischemic regions and differentiated in ischemic boundaries into doublecortin+ neuroblasts at higher rates in animals with MSC transplantation compared to control groups. The study indicates that therapeutic effects of MSCs can be at least partly ascribed to the dual functions of MSCs including enhancing endogenous neurogenesis and protecting nascent cells from a deleterious environment [81].

There may also be an interaction between injected stem cells and the microglia, in which the microglia may act as a key factor in stimulation of proliferation of endogenous neural stem cells. For example, following 2 hour middle cerebral artery occlusion in rats, one study observed increased numbers of activated microglia in the ipsilateral subventricular zone (SVZ) concomitant with neuroblast migration into the striatum at 2, 6, and 16 weeks, with maximum migration at 6 weeks. In the peri-infarct striatum, numbers of activated microglia peaked at 2 weeks and declined thereafter. Microglia in the SVZ were resident or originated from bone marrow, with maximum proliferation during the first 2 weeks post-insult. In the SVZ, microglia exhibited ramified or intermediate morphology, signifying a downregulated inflammatory profile, whereas amoeboid or round phagocytic microglia were frequent in the peri-infarct striatum. Numbers of microglia expressing markers of antigen-presenting cells (MHC-II, CD86) increased in the SVZ, but very few lymphocytes were detected. Using quantitative PCR, a strong short- and long-term increase (at 1 and 6 weeks post-infarct) of insulin-like growth factor-1 (IGF-1) gene expression was detected in SVZ tissue. Elevated numbers of IGF-1-expressing microglia were found in the SVZ at 2, 6, and 16 weeks after stroke. At 16 weeks, 5% of microglia but no other cells in the SVZ expressed the IGF-1 protein, which mitigates apoptosis and promotes proliferation and differentiation of neural stem cells (NSCs). The long-term accumulation of microglia with a pro-neurogenic phenotype in the SVZ implies a supportive role for these cells in continuous neurogenesis after stroke [82].

Additional support for stimulation of endogenous neurogenesis by exogenously administered stem cells comes from a study in which scientists tested the hypothesis that neurotrophic factors secreted by human bone marrow-derived MSCs (hBMSCs) promote endogenous neurogenesis, reduce apoptosis, and improve functional recovery. Adult rats subjected to 2 hour middle cerebral artery occlusion (MCAO) were transplanted with hBMSCs or saline into the ipsilateral brain parenchyma at 3 days after ischemia. There was a significant recovery of behavior in the hBMSCs-treated rats beginning at 14 days after MCAO compared with the control animals. Higher levels of brain-derived neurotrophic factor (BDNF), neurotrophin-3 (NT-3), and vascular endothelial growth factor (VEGF) were detected in the hBMSC-treated rat brain compared to the control. Human BMSC treatment also enhanced endogenous cell proliferation both in the SVZ and in the subgranular zone (SGZ) of the hippocampus. In addition, in rats treated with hBMSCs, more neuronal progenitor cells migrated from the SVZ to the ischemic boundary zone (IBZ) and differentiated into mature neurons with less apoptosis. Overall, these data suggest an essential role for hBMSCs in promoting endogenous neurogenesis, protecting newly formed cells, and improving functional recovery after ischemia in rats [83].

Interestingly, the relationship between bone marrow and stroke may be bidirectional. In one study, it was shown that stroke actually manipulates the hematopoietic stem cell compartment. Specifically, serial in vivo bioluminescence reporter gene imaging in mice with transient cerebral middle artery occlusion (tMCAO) revealed that bone marrow cell cycling peaked 4 days after stroke (P<0.05 versus pre-tMCAO). Flow cytometry and cell cycle analysis showed activation of the entire hematopoietic tree, including myeloid progenitors. The cycling fraction of the most upstream hematopoietic stem cells increased from 3.34% ± 0.19% to 7.32% ± 0.52% after tMCAO (P<0.05). In vivo microscopy corroborated proliferation of adoptively transferred hematopoietic progenitors in the bone marrow of mice with stroke. The hematopoietic system’s myeloid bias was reflected by increased expression of myeloid transcription factors, including PU.1 (P<0.05), and by a decline in lymphocyte precursors. In mice after tMCAO, tyrosine hydroxylase levels in sympathetic fibers and bone marrow noradrenaline levels rose (P<0.05, respectively), and were associated with a decrease in hematopoietic niche factors that promote stem cell quiescence. In mice with genetic deficiency of the β3 adrenergic receptor, hematopoietic stem cells did not enter the cell cycle in increased numbers after tMCAO (naive control, 3.23 ± 0.22; tMCAO, 3.74 ± 0.33, P=0.51) [84].

Despite the interesting animal studies described above, direct evidence correlating increased regional cerebral blood flow and functional recovery has yet to be demonstrated in humans. Using ex vivo co-culture systems, possible interactions between angiogenesis and neurogenesis have been examined in, for example, three-dimensional culture models, including neurons, endothelial cells, and extracellular matrices. In one set of experiments, this was demonstrated by constructing 3D neurovascular tissues by combining in vitro neurogenesis and angiogenesis models using a microfluidic platform, which is a critical step toward neurovascular unit (NVU) construction in vitro. Three gel conditions, which were fibrin gel, fibrin-Matrigel mixed gel, and fibrin-hyaluronan mixed gel, were investigated to optimize the gel components in terms of neurogenesis and angiogenesis. First, fibrin-Matrigel mixed gel was found to promote neural stem cell (NSC) differentiation into neurons and neurite extensions. In particular, 3D neural networks were constructed in 2-8 mg/ml fibrin-Matrigel mixed gel. Second, investigators found that capillary-like structures were also formed in the fibrin-Matrigel mixed gel by co-culturing brain microvascular endothelial cells (BMECs) and human mesenchymal stem cells (MSCs). Finally, the researchers combined both neural and vascular culture models and succeeded in constructing 3D neurovascular tissues with an optimized seeding condition of NSCs, BMECs, and MSCs [85].

Other studies have shown that co-culture of endothelial cells with neural stem cells leads to expanded neurogenesis. Specifically, it was shown that endothelial cells but not vascular smooth muscle cells release soluble factors that stimulate the self-renewal of neural stem cells, inhibit their differentiation, and enhance their neuron production. Both embryonic and adult neural stem cells respond, allowing extensive production of both projection neurons and interneuron types in vitro. Endothelial co-culture stimulates neuroepithelial cell contact, activating Notch and Hes 1 to promote self-renewal. These findings indicate that endothelial cells are a critical component of the neural stem cell niche [86].

The critical importance and contribution of endothelial cells to the neural stem cell niche has been reported by other studies as well. In one study, researchers analyzed expression at the protein level of a panel of angiogenic and/or neurotrophic factors and their receptors in the SVZa of adult macaque monkeys under normal conditions or after transient global ischemia, which enhances endogenous progenitor cell proliferation. The investigators found that fms-like tyrosine kinase 1 (Flt1), a receptor for vascular endothelial cell growth factor, was expressed by over 30% of the proliferating progenitors, and the number of Flt1-positive precursors was significantly increased by the ischemic insult. Smaller fractions of mitotic progenitors were positive for the neurotrophin receptor tropomyosin-related kinase (Trk) B or the hematopoietic receptor Kit, while immature neurons expressed Flt1 and the neurotrophin receptor TrkA. Further, SVZa astroglia, ependymal cells, and blood vessels were positive for distinctive sets of ligands/receptors, which were characterized. The data provide a molecular phenotypic analysis of the cell types comprising the adult monkey SVZa and suggest that a complex network of angiogenic/neurotrophic signals operating in an autocrine or paracrine manner may regulate SVZa neurogenesis in the adult primate brain [87].

It appears in some cases that injury to endothelial cells actually modulates the effects of neural stem cells. For example, in one study, intact or oxygen-glucose deprived (OGD) endothelial cells secreted factors that enhanced neurogenesis. The researchers co-cultured mouse SVZ neurospheres (NS) with endothelial cells or differentiated NS in endothelial cell-conditioned medium (ECCM). NS also were expanded in ECCM from OGD-exposed (OGD-ECCM) endothelial cells to assess injury effects. ECCM significantly increased NS production. NS co-cultured with endothelial cells or ECCM generated more immature-appearing neurons and oligodendrocytes, and astrocytes with radial glial-like/reactive morphology than controls. OGD-ECCM stimulated neuroblast migration and yielded neurons with longer processes and more branching. These data indicate that intact and injured endothelial cells exert differing effects on neural stem cells and suggest targets for stimulating regeneration of neurons after brain insults [88].

Angiogenic factors are known to produce neurogenesis. For example, Angiopoietin-1 (Ang-1) is an endothelial growth factor with a critical role in division, survival, and adhesion of endothelial cells via Tie-2 receptor activity. Expression of Tie-2 in non-endothelial cells, especially neurons and stem cells, suggests that Ang-1 may be involved in neurogenesis. In one study, researchers investigated the putative role of Ang-1 on SVZ neurogenesis. Immature cells from SVZ-derived neurospheres express Ang-1 and Tie-2 mRNA, suggesting a role for the Ang-⅟Tie-2 system in the neurogenic niche. Moreover, it was also found that Tie-2 protein expression is retained on differentiation in neurons and glial cells. Ang-1 triggered proliferation via activation of the ERK½ (extracellular signal-regulated kinase ½) mitogen-activated protein kinase (MAPK) kinase pathway but did not induce cell death. Accordingly, co-incubation with an anti-Tie-2 neutralizing antibody prevented the pro-proliferative effect of Ang-1. Furthermore, Ang-1 increased the number of NeuN (neuronal nuclear protein)-positive neurons in cultures treated for 7 days, as well as the number of functional neurons, as assessed by monitoring [Ca(2+)](i) rises after application of specific stimuli for neurons and immature cells. The pro-neurogenic effect of Ang-1 is mediated by Tie-2 activation and subsequent mTOR (mammalian target of rapamycin kinase) mobilization. In agreement, neuronal differentiation significantly decreased after exposure to an anti-Tie-2 neutralizing antibody and to rapamycin. Moreover, Ang-1 elicited the activation of the SAPK (stress-activated protein kinase)/JNK (c-Jun N-terminal kinase) MAPK, involved in axonogenesis. This work shows a proneurogenic effect of Ang-1, highlighting the relevance of blood vessel/stem cell cross talk in health and disease [89].

Other studies have shown that conditioned media from endothelial cells protected neuronal cells against oxygen-glucose deprivation via brain-derived neurotrophic factor (BDNF). Specifically, investigators showed that the cerebral endothelium is not comprised of inert tubes for delivering blood. Instead, they also secrete trophic factors that can be directly neuroprotective. Conditioned media from cerebral endothelial cells broadly protects neurons against oxygen-glucose deprivation, oxidative damage, endoplasmic reticulum stress, hypoxia, and amyloid neurotoxicity. This phenomenon is largely mediated by endothelial-produced brain-derived neurotrophic factor (BDNF) because filtering endothelial-conditioned media with TrkB-Fc, which inhibits BDNF-induced phosphorylation of ERK½, eliminates the neuroprotective effect. Endothelial production of BDNF is sustained by β-1 integrin and integrin-linked kinase (ILK) signaling. Non-cytotoxic levels of oxidative stress disrupts ILK signaling and reduces endothelial levels of neuroprotective BDNF. These data suggest that the cerebral endothelium provides a critical source of homeostatic support for neurons [90].

Indeed, several mediators have been shown to modulate both angiogenesis and axonal outgrowth and communicate with cells within the affected neurovascular units. These include vascular endothelial growth factor (VEGF) [91-109], transforming growth factor-β [110], angiopoetin-1 [111, 112], platelet-derived growth factor-B [113-119], BDNF [120-124], GDF-11 [125, 126], and progranulin [127]. In addition, blood vessels secrete signals (e.g., VEGF, artemin, and neurotrophin) to guide axons, and conversely, axons secrete signals to guide blood vessels. As such, each cell in the neurovascular unit can communicate with other cells using various mediators.

One embodiment of the present disclosure encompasses the use of a probiotic/enzyme mixture to enhance regenerative cell chemotaxis to an area of infarct. One means by which regenerative cells can enter areas of ischemia is through mobilization based on chemotactic gradients. In one study using a stroke model, the chemotactic gradient SDF-1 was examined in the brains of 40 mice relative to the homing of bone marrow-derived cells to sites of ischemic injury. Mice received bone marrow transplants from green fluorescent protein (GFP) transgenic donors and later underwent a temporary middle cerebral artery suture occlusion (MCAO). SDF-1 was associated with blood vessels and cellular profiles by 24 hours through at least 30 days post-MCAO. SDF-1 expression was principally localized to the ischemic penumbra. The majority of SDF-1 expression was associated with reactive astrocytes; much of this was perivascular. GFP+ cells were associated with SDF-1-positive vessels and were also found in the neuropil of regions with increased SDF-1 immunoreactivity. Most vessel-associated GFP+ cells resembled pericytes or perivascular microglia, and the majority of the GFP+ cells in the parenchyma displayed characteristics of activated microglial cells. These findings suggest SDF-1 is important in the homing of bone marrow-derived cells, especially monocytes, to areas of ischemic injury [33].

In one embodiment, a probiotic/enzyme mixture is used to augment T regulatory (T(reg)) cell numbers and activity in order to reduce stroke pathology or other central nervous system pathologies. Depletion of T(reg) cells profoundly increases delayed brain damage and deteriorated functional outcome. An absence of T(reg) cells augments post-ischemic activation of resident and invading inflammatory cells including microglia and T cells, the main sources of deleterious cerebral tumor necrosis factor-α (TNF-α) and interferon-γ (IFN-γ), respectively. Early antagonization of TNF-α and delayed neutralization of IFN-γ prevents infarct growth in T(reg) cell-depleted mice. Intracerebral interleukin-10 (IL-10) substitution abrogates cytokine overexpression after T(reg) cell depletion and prevents secondary infarct growth, whereas transfer of IL-10-deficient T(reg) cells in an adoptive transfer model is ineffective. In conclusion, T(reg) cells are major cerebroprotective modulators of post-ischemic inflammatory brain damage, targeting multiple inflammatory pathways. IL-10 signaling is essential for the immunomodulatory effect of T(reg) cells. In some embodiments, treatment with the fibroblasts of the present disclosure can inhibit production of IL-10.

In one embodiment, the present disclosure encompasses the use of a probiotic/enzyme mixture as a treatment to enhance the efficacy of regenerative-based approaches. The use of regenerative-based approaches for treatment of stroke has been previously described. For instance, one of the first publications regarding cerebral infarcts treated by MSC was a case report on a 17-year-old Korean man diagnosed with basilar artery dissection. Infarction of the bilateral pons, midbrain, and right superior cerebellum due to his basilar artery dissection was partially re-canalized by intrathecal injection of human umbilical cord blood-derived mesenchymal stem cells. No immunosuppressants were given to the patient, and human leukocyte antigen alloantibodies were not detected after cell therapy. A subjective response was reported [132].

Another study involved prospectively and randomly allocation of 30 patients with cerebral infarcts within the middle cerebral arterial territory and with severe neurological deficits into one of two treatment groups: an MSC group (n = 5) received intravenous infusion of 1 × 10(8) autologous MSCs, whereas a control group (n = 25) did not receive MSCs. Changes in neurological deficits and improvements in function were compared between the groups for 1 year after symptom onset. Neuroimaging was performed serially in five patients from each group. Outcomes improved in the MSC-treated patients compared with the control patients: the Barthel index (p = 0.011, 0.017, and 0.115 at 3, 6, and 12 months, respectively) and modified Rankin score (p = 0.076, 0.171, and 0.286 at 3, 6, and 12 months, respectively) of the MSC group improved consistently during the follow-up period. Serial evaluations showed no adverse cell-related, serological, or imaging-defined effects. The authors concluded that for patients with severe cerebral infarcts, intravenous infusion of autologous MSCs appears to be a feasible and safe therapy that may improve functional recovery [128].

A follow up to this study by the same group reported on an open-label, observer-blinded clinical trial of 85 patients with severe middle cerebral artery territory infarct. Patients were randomly allocated to one of two groups, those who received i.v. autologous ex vivo cultured MSCs (MSC group) or those who did not (control group), and followed for up to 5 years. Mortality of any cause, long-term side effects, and new-onset comorbidities were monitored. Of the 52 patients who were finally included in this study, 16 were assigned to the MSC group and 36 were assigned to the control group. Four (25%) patients in the MSC group and 21 (58.3%) in the control group died during the follow-up period, and the cumulative surviving portion at 260 weeks was 0.72 in the MSC group and 0.34 in the control group (log-rank; p = .058). Significant side effects were not observed following MSC treatment. The occurrence of comorbidities including seizures and recurrent vascular episodes did not differ between groups. When compared with the control group, the follow-up modified Rankin Scale (mRS) score was decreased, whereas the number of patients with a mRS of 0-3 increased in the MSC group (p = .046). Clinical improvement in the MSC group was associated with serum levels of stromal cell-derived factor-1 and involvement of the subventricular region of the lateral ventricle. Intravenous autologous MSCs transplantation was safe for stroke patients during long-term follow-up. This therapy may improve recovery after stroke depending on specific patient characteristics [129].

Another study used autologous BM-MSC in 12 patients who had stroke lasting 3 months to 1 year and NIH Stroke Scale/Scores (NIHSS) of 4-15. Patients had to be conscious and able to comprehend. Fugl Meyer (FM), modified Barthel index (mBI), MRC, Ashworth tone grade scale scores, and functional imaging scans were assessed at baseline, after 8 weeks, and after 24 weeks. Bone marrow was aspirated under aseptic conditions, and MSC were expanded for 3 weeks with animal serum-free media (Stem Pro SFM). Six patients were administered a mean of 50-60 × 10(6) cells i.v. followed by 8 weeks of physiotherapy. Six patients served as controls. This was a non-randomized experimental controlled trial. Clinical and radiological scanning was normal for the stem cell group patients. There was no mortality or cell-related adverse reaction. The laboratory tests on days 1, 3, 5 and 7 were also normal in the MSC group until the last follow-up. The FM and mBI showed a modest increase in the stem cell group compared to controls. There was an increased number of cluster activation of Brodmann areas BA 4 and BA 6 after stem cell infusion compared to controls, indicating neural plasticity [130].

A similar study used patients’ own sera for expansion of autologous BM-MSC. The publication described an unblinded study of 12 patients with ischemic grey matter, white matter, and mixed lesions, in contrast to a prior study on autologous mesenchymal stem cells expanded in fetal calf serum that focused on grey matter lesions. Cells cultured in human serum expanded more rapidly than in fetal calf serum, reducing cell preparation time and the risk of transmissible disorders such as bovine spongiform encephalomyelitis. Autologous mesenchymal stem cells were delivered intravenously 36-133 days post-stroke. All patients had magnetic resonance angiography to identify vascular lesions and magnetic resonance imaging prior to cell infusion and at intervals up to 1 year after. Magnetic resonance perfusion-imaging and 3D-tractography were carried out in some patients. Neurological status was scored using the NIHSS and modified Rankin scores. Researchers did not observe any central nervous system tumors, abnormal cell growths, or neurological deterioration, and there was no evidence for venous thromboembolism, systemic malignancy, or systemic infection in any patients following stem cell infusion. The median daily rate of NIHSS change was 0.36 during the first week post-infusion, compared with a median daily rate of change of 0.04 from the first day of testing to immediately before infusion. Daily rates of change in NIHSS scores during longer post-infusion intervals that more closely matched the interval between initial scoring and cell infusion also showed an increase following cell infusion. Mean lesion volume as assessed by magnetic resonance imaging was reduced by >20% at 1 week post-cell infusion [131].

Support for use of probiotics to augment therapeutic processes induced by regenerative fibroblasts can be found in studies for treatment of several disease conditions. For example, in the treatment of graft versus host disease (GVHD), GVHD has been associated with dysbiosis [133-139]. Studies have shown some potential effects of probiotics to stimulate Treg cells [140-154], and Tregs inhibit GVHD [155-172]. Using regenerative fibroblasts to enhance Treg activity and to prevent GVHD has not been studied, however.

Another embodiment of the present disclosure provides for probiotic treatment prior to utilization of regenerative fibroblasts to reduce restenosis after stent placement. It is known that stent placement causes chronic inflammation [173-175], and chronic inflammation causes restenosis [176-178]. Accordingly, the use of probiotics to repair the microbiome is envisioned prior to treatment with regenerative fibroblasts to enhance endothelialization and reduce inflammation after stent placement.

Spinal cord injury is also associated with dysbiosis [53-56], and probiotics have been shown to be effective in treating animal models of spinal cord injury [57, 58]. Accordingly, in one embodiment, probiotics are administered to repair dysbiosis before regenerative fibroblasts are administered as a therapeutic intervention.

Liver failure associated with dysbiosis [59, 60]. Liver failure associated with inflammation [61-63]. Probiotics inhibit progression of liver failure [64, 65]. Heart failure associated with dysbiosis [66-68].

Autism is associated with an inflammatory profile [69-97] and reduced production of anti-inflammatory agents [98, 99]. Further, autistic subjects have been shown to have dysbiosis [100-109]. Probiotics have demonstrated positive effects in treating animal models of autism [110-113]

### VI. Administration of Therapeutic Compositions

The therapy provided herein may comprise administration of a therapeutic agents (e.g., fibroblasts, exosomes from fibroblasts, other fibroblast-derived products or fibroblast-produced therapeutic factors, etc.) alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents (e.g., fibroblasts) of the disclosure may be administered by the same route of administration or by different routes of administration. In some embodiments, the cancer therapy is administered intravenously, intramuscularly, subcutaneously, topically, orally, sublingually, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual’s clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, subjects are treated with fibroblasts within a composition. Fibroblasts can be activated prior to therapeutic use and/or fibroblasts can be administered with agents which act as “regenerative adjuvants” for the fibroblasts. In some embodiments, compositions containing fibroblasts comprise between about 10,000-10 million cells per kilogram of body weight. In some embodiments, approximately 1 million cells per kilogram of body weight are administered. In some embodiments, between about 10⁵ and about 10¹³ cells per 100 kg are administered to a human per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹¹ cells are infused per 100 kg. In some embodiments, 100 kg of body weight is measured as 100 kg of body weight of a human.. In some embodiments, between about 50 million and about 500 million fibroblast cells are administered to the subject. For example, between about 50 million and about 100 million fibroblast cells, between about 50 million and about 200 million fibroblast cells, between about 50 million and about 300 million fibroblast cells, between about 50 million and about 400 million fibroblast cells, between about 100 million and about 200 million fibroblast cells, between about 100 million and about 300 million fibroblast cells, between about 100 million and about 400 million fibroblast cells, between about 100 million and about 500 million fibroblast cells, between about 200 million and about 300 million fibroblast cells, between about 200 million and about 400 million fibroblast cells, between about 200 million and about 500 million fibroblast cells, between about 300 million and about 400 million fibroblast cells, between about 300 million and about 500 million fibroblast cells, between about 400 million and about 500 million fibroblast cells, about 50 million fibroblast cells, about 100 million fibroblast cells, about 150 million fibroblast cells, about 200 million fibroblast cells, about 250 million fibroblast cells, about 300 million fibroblast cells, about 350 million fibroblast cells, about 400 million fibroblast cells, about 450 million fibroblast cells, or about 500 million fibroblast cells may be administered to the subject.

In some embodiments, a single administration of cells is provided. The cells can be fibroblasts and/or other cells of the disclosure. In some embodiments, cells are administered once monthly, weekly, and/or daily. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹⁰ cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days. In some embodiments, up to about 20 administrations of between about 10⁵ and about 10¹³ cells per 100 kg are provided.

The quantity to be administered, both according to number of treatments and unit dose, depends on the treatment effect desired. An effective dose is understood to refer to an amount necessary to achieve a particular effect. In some embodiments, it is contemplated that doses between about 10⁵ and about 10¹³ cells per 100 kg are administered to affect the protective capability of the fibroblasts. In certain embodiments, it is contemplated that a cell number in the range from 10 mg/kg to 200 mg/kg can affect the protective capability of these agents. Thus, it is contemplated that doses include doses of cell numbers of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 µg/kg, mg/kg, µg/day, or mg/day or any range derivable therein. Furthermore, such doses can be administered at multiple times during a day, and/or on multiple days, weeks, or months.

In some embodiments, it is contemplated that a dose of a drug or other therapeutic agent in the range from 10 mg/kg to 200 mg/kg can affect the protective capability of these agents. Thus, it is contemplated that doses include doses of drugs or other therapeutic agents of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 µg/kg, mg/kg, µg/day, or mg/day or any range derivable therein. Furthermore, such doses can be administered at multiple times during a day, and/or on multiple days, weeks, or months.

It will be understood by those skilled in the art and made aware that dosage units of µg/kg or mg/kg of body weight can be converted and expressed in comparable concentration units of µg/ml or mM (blood levels), such as 4 µM to 100 µM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain embodiments, the effective dose of a pharmaceutical composition is one which can provide a blood level of about 1 µM to 150 µM. In another embodiment, the effective dose provides a blood level of about 4 µM to 100 µM.; or about 1 µM to 100 µM; or about 1 µM to 50 µM; or about 1 µM to 40 µM; or about 1 µM to 30 µM; or about 1 µM to 20 µM; or about 1 µM to 10 µM; or about 10 µM to 150 µM; or about 10 µM to 100 µM; or about 10 µM to 50 µM; or about 25 µM to 150 µM; or about 25 µM to 100 µM; or about 25 µM to 50 µM; or about 50 µM to 150 µM; or about 50 µM to 100 µM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 µM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

### VII. Kits of the Disclosure

Any of the cellular and/or non-cellular compositions described herein or similar thereto may be comprised in a kit. In a non-limiting example, one or more reagents for use in methods for preparing fibroblasts or derivatives thereof may be comprised in a kit. Such reagents may include cells, vectors, one or more growth factors, vector(s), one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### EXAMPLES

The following examples are included to demonstrate particular embodiments of the invention. It should be appreciated by those of skill in the art that the techniques disclosed in the examples that follow represent techniques discovered by the inventors to function well in the practice of the methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

Female BALB/c mice at eight weeks of age were treated for 7 days with dextran sodium sulfate (DSS) at 5% in their drinking water in order to induce a murine version of inflammatory bowel disease (IBD).

Mice were divided into groups of 10 mice per group. Group 1 mice received DSS daily; Group 2 mice received DSS and fibroblasts (500,000 cells intravenously on days 1 and 7); Group 3 mice received DSS and 400 uL saline gavage containing 10⁸ colony forming units of Bifidobacterium infantis; Group 4 mice recieved DSS, 400 uL saline gavage containing 10⁸ colony forming units of Bifidobacterium infantis, and fibroblasts (500,000 cells intravenously on days 1 and 3).

Disease activity index (DAI) scores reflecting the general condition of the mice were calculated according to Table 1 below (Hamamoto et al. Clin Exp Immunol. 1999 Sep; 117(3):462-8). Briefly, animals were weighed before starting DSS administration and on the day when animals were killed. Stool consistency and the degree of blood in stool were evaluated on the day when animals were killed. Simultaneously, hemoglobin levels were examined in intracardiac blood. (DAI) = (combined score of weight loss, stool consistency, and bleeding)/3. Normal stools was defined as well-formed pellets; loose stools was defined as pasty stools that do not stick to the anus; and diarrhea was defined as liquid stools that stick to the anus.

TABLE 1

Disease Activity Index

| Score | Weight loss (%) | Stool consistency | Visible blood in feces |
|---|---|---|---|
| 0 | None | Normal | None |
| 1 | 1-5 | | |
| 2 | 6-10 | Loose | Slight bleeding |
| 3 | 11-20 | | |
| 4 | >20 | Diarrhea | Gross bleeding |

### Example 2

Female BALB/c mice at eight weeks of age were treated for 7 days with dextran sodium sulfate (DSS) at 5% in their drinking water in order to induce a murine version of inflammatory bowel disease (IBD).

Mice were divided into groups of 10 mice per group. Group 1 mice received DSS daily; Group 2 mice received DSS and fibroblasts (500,000 cells intravenously on days 1 and 7); Group 3 mice received DSS and 400 uL saline gavage containing 10⁸ colony forming units of Bifidobacterium infantis; Group 4 mice recieved DSS, 400 uL saline gavage containing 10⁸ colony forming units of Bifidobacterium infantis, and fibroblasts (500,000 cells intravenously on days 1 and 3).

Mice were sacrificed on days 3, 5, and 7, and assessment of IL-6 in plasma was performed using an IL-6 ELISA kit.

### Example 3

### Induction of Experimental Allergic Encephalomyelitis (EAE)

Primary progressive EAE model for C57BL/6 mice was established by immunizing subcutaneously on the back with 0.2 mL of myelin oligodendrocyte glycoprotein (MOG) 35-55 peptide (MEVGWYRSPFSRVVHLYRNGK (SEQ ID NO:1), HPLC-purity: >95%) emulsified in complete Freund’s adjuvant (CFA) containing 4 mg/mL Mycobacterium tuberculosis H37Ra. These injections were distributed over the following three sites: one along the midline of the back between the shoulders and two on either side of the midline on the lower back. The final dose of MOG 35-55 and Mycobacterium tuberculosis H37Ra was 200 µg and 400 µg per mouse. Each mouse received an additional 400 ng of pertussis toxin by intraperitoneal injection of 200 µL PBS on day 0 and day 2 post-immunization. Clinical scores were calculated blindly by two researchers daily according to a 0-5 scale as follows 1, limp tail or waddling gait with tail tonicity; 2, waddling gait with limp tail (ataxia); 2.5, ataxia with partial limb paralysis; 3, full paralysis of 1 limb; 3.5, full paralysis of one limb with partial paralysis of the second limb; 4, full paralysis of two limbs; 4.5, moribund; and 5, death.

### Induction of Dysbiosis

Two weeks prior to initial immunization, Neosulfox (Sulfadimidine sodium 10% (w/w), neomycin sulfate 6%, oxytetracycline hydrochloride 4%, 2.5 g/l and Pentrexyl (ampicillin) were applied in drinking water and changed regularly every second day. Antibiotics were given throughout the length of the experiment until termination at day 30.

### Administration of Fibroblasts

Foreskin fibroblasts were selected for CD73 expression using Magnetic Activated Sorting and administered at a concentration of 500,000 cells intravenously on day 2 post immunization.

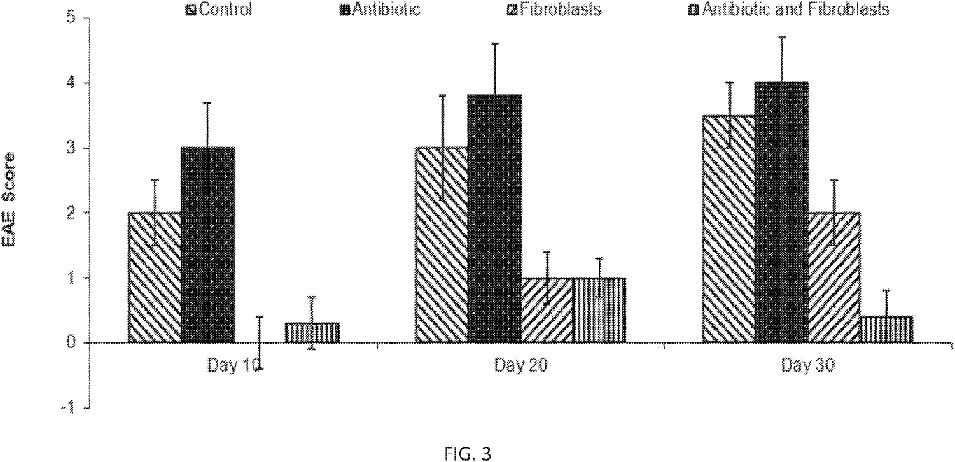

FIG. 3 demonstrates EAE scores in which fibroblasts and the combination of fibroblasts and antibiotic improved the EAE scores, reflecting an enhancement of the manipulation of the microbiome.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

1. Krupinski, J., et al., Role of angiogenesis in patients with cerebral ischemic stroke. Stroke, 1994. 25(9): p. 1794-8.

2. Schneider, U.C., et al., Granulocyte-macrophage colony-stimulating factor-inducedvessel growth restores cerebral blood supply after bilateral carotid artery occlusion. Stroke, 2007. 38(4): p. 1320-8.

3. Ardelt, A.A., et al., Estradiol augments peri-infarct cerebral vascular density inexperimental stroke. Exp Neurol, 2007. 206(1): p. 95-100.

4. Arai, K., et al., Brain angiogenesis in developmental and pathological processes:neurovascular injury and angiogenic recovery after stroke. FEBS J, 2009. 276(17): p. 4644-52.

5. Jin, Y., et al., Poststroke Sonic Hedgehog Agonist Treatment Improves FunctionalRecovery by Enhancing Neurogenesis and Angiogenesis. Stroke, 2017. 48(6): p. 1636-1645.

6. Kanazawa, M., et al., Angiogenesis in the ischemic core: A potential treatment target? J Cereb Blood Flow Metab, 2019. 39(5): p. 753-769.

7. Marushima, A., et al., Balanced single-vector co-delivery of VEGF/PDGF-BBimproves functional collateralization in chronic cerebral ischemia. J Cereb Blood Flow Metab, 2019: p. 271678X18818298.

8. Iyer, N.V., et al., Cellular and developmental control of O2 homeostasis by hypoxia-inducible factor 1 alpha. Genes Dev, 1998. 12(2): p. 149-62.

9. Bergeron, M., et al., Induction of hypoxia-inducible factor-1 (HIF-1) and its target genes following focal ischaemia in rat brain. Eur J Neurosci, 1999. 11(12): p. 4159-70.

10. Zhang, Z., et al., Hypoxia inducible factor-1 as a target for neurodegenerative diseases. Curr Med Chem, 2011. 18(28): p. 4335-43.

11. Liu, B.N., B.X. Han, and F. Liu, Neuroprotective effect of pAkt and HIF-1 alpha on ischemia rats. Asian Pac J Trop Med, 2014. 7(3): p. 221-5.

12. Huang, T., et al., Hypoxia-inducible factor-1alpha upregulation in microglia following hypoxia protects against ischemia-induced cerebral infarction. Neuroreport, 2014. 25(14): p. 1122-8.

13. Kumar, H. and D.K. Choi, Hypoxia Inducible Factor Pathway and Physiological Adaptation: A Cell Survival Pathway? Mediators Inflamm, 2015. 2015: p. 584758.

14. Ceradini, D.J., et al., Progenitor cell trafficking is regulated by hypoxic gradients through HIF-1 induction of SDF-1. Nat Med, 2004. 10(8): p. 858-64.

15. Ceradini, D.J. and G.C. Gurtner, Homing to hypoxia: HIF-1 as a mediator of progenitor cell recruitment to injured tissue. Trends Cardiovasc Med, 2005. 15(2): p. 57-63.

16. Tabatabai, G., et al., Irradiation and hypoxia promote homing of haematopoietic progenitor cells towards gliomas by TGF-beta-dependent HIF-1 alpha-mediated induction of CXCL12. Brain, 2006. 129(Pt 9): p. 2426-35.

17. Chang, Y.C., et al., Regenerative therapy for stroke. Cell Transplant, 2007. 16(2): p. 171-81.

18. Hoenig, M.R., C. Bianchi, and F.W. Sellke, Hypoxia inducible factor-1 alpha, endothelial progenitor cells, monocytes, cardiovascular risk, wound healing, cobalt and hydralazine: a unifying hypothesis. Curr Drug Targets, 2008. 9(5): p. 422-35.

19. Marti, H.J., et al., Hypoxia-induced vascular endothelial growth factor expression precedes neovascularization after cerebral ischemia. Am J Pathol, 2000. 156(3): p. 965-76.

20. Harms, K.M., L. Li, and L.A. Cunningham, Murine neural stem/progenitor cells protect neurons against ischemia by HIF-1 alpha-regulated VEGF signaling. PLoS One, 2010. 5(3): p. e9767.

21. Cunningham, L.A., K. Candelario, and L. Li, Roles for HIF-1alpha in neural stem cell function and the regenerative response to stroke. Behav Brain Res, 2012. 227(2): p. 410-7.

22. Lai, T.W., et al., HIF-1alpha binding to the Epac1 promoter recruits hematopoietic stem cells to the ischemic brain following stroke. J Mol Cell Biol, 2012. 4(3): p. 184-7.

23. Hu, Q., et al., Delayed hyperbaric oxygen therapy promotes neurogenesis through reactive oxygen species/hypoxia-inducible factor-1alpha/beta-catenin pathway in middle cerebral artery occlusion rats. Stroke, 2014. 45(6): p. 1807-14.

24. Khan, M., et al., Promoting endothelial function by S-nitrosoglutathione through the HIF-1 alpha/VEGF pathway stimulates neurorepair and functional recovery following experimental stroke in rats. Drug Des Devel Ther, 2015. 9: p. 2233-47.

25. Badawi, Y., P. Ramamoorthy, and H. Shi, Hypoxia-inducible factor 1 protects hypoxic astrocytes against glutamate toxicity. ASN Neuro, 2012. 4(4): p. 231-41.

26. Chen, J., Y. Li, and M. Chopp, Intracerebral transplantation of bone marrow with BDNF after MCAo in rat. Neuropharmacology, 2000. 39(5): p. 711-6.

27. Li, Y., J. Chen, and M. Chopp, Adult bone marrow transplantation after stroke in adult rats. Cell Transplant, 2001. 10(1): p. 31-40.

28. Li, Y., et al., Treatment of stroke in rat with intracarotid administration of marrow stromal cells. Neurology, 2001. 56(12): p. 1666-72.

29. Zhang, Z.G., et al., Bone marrow-derived endothelial progenitor cells participate in cerebral neovascularization after focal cerebral ischemia in the adult mouse. Circ Res, 2002. 90(3): p. 284-8.

30. Zhao, L.R., et al., Human bone marrow stem cells exhibit neural phenotypes and ameliorate neurological deficits after grafting into the ischemic brain of rats. Exp Neurol, 2002. 174(1): p. 11-20.

31. Hess, D.C., et al., Bone marrow as a source of endothelial cells and NeuN-expressing cells After stroke. Stroke, 2002. 33(5): p. 1362-8.

32. Willing, A.E., et al., Mobilized peripheral blood cells administered intravenously produce functional recovery in stroke. Cell Transplant, 2003. 12(4): p. 449-54.

33. Hill, W.D., et al., SDF-1 (CXCL12) is upregulated in the ischemic penumbra following stroke: association with bone marrow cell homing to injury. J Neuropathol Exp Neurol, 2004. 63(1): p. 84-96.

34. Shyu, W.C., et al., Functional recovery of stroke rats induced by granulocyte colony-stimulating factor-stimulated stem cells. Circulation, 2004. 110(13): p. 1847-54.

35. Shen, L.H., et al., Intracarotid transplantation of bone marrow stromal cells increases axon-myelin remodeling after stroke. Neuroscience, 2006. 137(2): p. 393-9.

36. Baker, A.H., et al., Brain protection using autologous bone marrow cell, metalloproteinase inhibitors, and metabolic treatment in cerebral ischemia. Proc Natl Acad Sci USA, 2007. 104(9): p. 3597-602.

37. Wu, J., et al., Intravenously Administered Bone Marrow Cells Migrate to Damaged Brain Tissue and Improve Neural Function in Ischemic Rats. Cell Transplant, 2007. 16(10): p. 993-1005.

38. Chen, J.R., et al., Transplanted bone marrow stromal cells migrate, differentiate and improve motor function in rats with experimentally induced cerebral stroke. J Anat, 2008. 213(3): p. 249-58.

39. Pavlichenko, N., et al., Mesenchymal stem cells transplantation could be beneficial for treatment of experimental ischemic stroke in rats. Brain Res, 2008. 1233: p. 203-13.

40. Omori, Y., et al., Optimization of a therapeutic protocol for intravenous injection of human mesenchymal stem cells after cerebral ischemia in adult rats. Brain Res, 2008. 1236: p. 30-8.

41. Chen, Z.Z., et al., Beneficial effect of autologous transplantation of bone marrow stromal cells and endothelial progenitor cells on cerebral ischemia in rabbits. Neurosci Lett, 2008. 445(1): p. 36-41.

42. Keimpema, E., et al., Early transient presence of implanted bone marrow stem cells reduces lesion size after cerebral ischaemia in adult rats. Neuropathol Appl Neurobiol, 2009. 35(1): p. 89-102.

43. Yasuhara, T., et al., Notch-induced rat and human bone marrow stromal cell grafts reduce ischemic cell loss and ameliorate behavioral deficits in chronic stroke animals. Stem Cells Dev, 2009. 18(10): p. 1501-14.

44. Deng, Y.B., et al., Intravenously administered BMSCs reduce neuronal apoptosis and promote neuronal proliferation through the release of VEGF after stroke in rats. Neurol Res, 2010. 32(2): p. 148-56.

45. Liu, N., et al., Expression of IL-10 and TNF-alpha in rats with cerebral infarction after transplantation with mesenchymal stem cells. Cell Mol Immunol, 2009. 6(3): p. 207-13.

46. Yang, M., et al., Changes in host blood factors and brain glia accompanying the functional recovery after systemic administration of bone marrow stem cells in ischemic stroke rats. Cell Transplant, 2010. 19(9): p. 1073-84.

47. Shen, L.H., Y. Li, and M. Chopp, Astrocytic endogenous glial cell derived neurotrophic factor production is enhanced by bone marrow stromal cell transplantation in the ischemic boundary zone after stroke in adult rats. Glia, 2010. 58(9): p. 1074-81.

48. Yilmaz, G., et al., Induction of neuro-protective/regenerative genes in stem cells infiltrating post-ischemic brain tissue. Exp Transl Stroke Med, 2010. 2(1): p. 11.

49. Felfly, H., et al., Hematopoietic stem cell transplantation protects mice from lethal stroke. Exp Neurol, 2010. 225(2): p. 284-93.

50. Zhang, X.M., et al., Transplanted bone marrow stem cells relocate to infarct penumbra and co-express endogenous proliferative and immature neuronal markers in a mouse model of ischemic cerebral stroke. BMC Neurosci, 2010. 11: p. 138.

51. Lowrance, S.A., et al., Bone-marrow-derived mesenchymal stem cells attenuate cognitive deficits in an endothelin-1 rat model of stroke. Restor Neurol Neurosci, 2015. 33(4): p. 579-88.

52. Yang, B., et al., Intra-arterial delivery is not superior to intravenous delivery of autologous bone marrow mononuclear cells in acute ischemic stroke. Stroke, 2013. 44(12): p. 3463-72.

53. Sharma, A., et al., Autologous bone marrow mononuclear cells in ischemic cerebrovascular accident paves way for neurorestoration: a case report. Case Rep Med, 2014.2014:p.530239.

54. Li, N., et al., Effect of bone marrow stromal cell transplantation on neurologic function and expression of VEGF in rats with focal cerebral ischemia. Mol Med Rep, 2014. 10(5): p. 2299-305.

55. Du, S., et al., Intra-arterial delivery of human bone marrow mesenchymal stem cells is a safe and effective way to treat cerebral ischemia in rats. Cell Transplant, 2014. 23 Suppl 1: p. S73-82.

56. Fukuda, Y., et al., Intra-arterial transplantation of low-dose stem cells provides functional recovery without adverse effects after stroke. Cell Mol Neurobiol, 2015. 35(3): p. 399-406.

57. Xu, Y., et al., Human bone marrow mesenchymal stem cell transplantation attenuates axonal injury in stroke rats. Neural Regen Res, 2014. 9(23): p. 2053-8.

58. Moniche, F., et al., Intra-arterial bone marrow mononuclear cells (BM-MNCs) transplantation in acute ischemic stroke (IBIS trial): protocol of a phase II, randomized, dose-finding, controlled multicenter trial. Int J Stroke, 2015. 10(7): p. 1149-52.

59. Yang, Z., et al., Bone marrow stromal cell transplantation through tail vein injection promotes angiogenesis and vascular endothelial growth factor expression in cerebral infarct area in rats. Cytotherapy, 2015. 17(9): p. 1200-12.

60. Lapi, D., et al., Effects of bone marrow mesenchymal stem cells (BM-MSCs) on rat pial microvascular remodeling after transient middle cerebral artery occlusion. Front Cell Neurosci, 2015. 9: p. 329.

61. Lv, W., et al., Bone marrow mesenchymal stem cells transplantation promotes the release of endogenous erythropoietin after ischemic stroke. Neural Regen Res, 2015. 10(8): p. 1265-70.

62. He, Q.Q., et al., Transplantation of bone marrow-derived mesenchymal stem cells (BMSCs) improves brain ischemia-induced pulmonary injury in rats associated to TNF-alpha expression. Behav Brain Funct, 2016. 12(1): p. 9.

63. Hu, J., et al., Bone marrow stromal cells inhibits HMGB1-mediated inflammation after stroke in type 2 diabetic rats. Neuroscience, 2016. 324: p. 11-9.

64. Yang, B., et al., Various Cell Populations Within the Mononuclear Fraction of Bone Marrow Contribute to the Beneficial Effects of Autologous Bone Marrow Cell Therapy in a Rodent Stroke Model. Transl Stroke Res, 2016. 7(4): p. 322-30.

65. Li, Y., et al., Neuroprotection by intravenous transplantation of bone marrow mononuclear cells from 5-fluorouracil pre-treated rats in a model of ischemic stroke. Neurol Res, 2016. 38(10): p. 921-8.

66. Sasaki, Y., et al., Synergic Effects of Rehabilitation and Intravenous Infusion of Mesenchymal Stem Cells After Stroke in Rats. Phys Ther, 2016. 96(11): p. 1791-1798.

67. Ghali, A.A., et al., Intra-arterial Infusion of Autologous Bone Marrow Mononuclear Stem Cells in Subacute Ischemic Stroke Patients. Front Neurol, 2016. 7: p. 228.

68. Garbuzova-Davis, S., et al., Intravenously Transplanted Human Bone Marrow Endothelial Progenitor Cells Engraft Within Brain Capillaries, Preserve Mitochondrial Morphology, and Display Pinocytotic Activity Toward Blood-Brain Barrier Repair in Ischemic Stroke Rats. Stem Cells, 2017. 35(5): p. 1246-1258.

69. Nakamura, H., et al., Elevated brain derived neurotrophic factor levels in plasma reflect in vivo functional viability of infused mesenchymal stem cells for stroke in rats. J Neurosurg Sci, 2019. 63(1): p. 42-49.

70. Yang, B., et al., Multipotent Adult Progenitor Cells Enhance Recovery After Stroke by Modulating the Immune Response from the Spleen. Stem Cells, 2017. 35(5): p. 1290-1302.

71. Sammali, E., et al., Intravenous infusion of human bone marrow mesenchymal stromal cells promotes functional recovery and neuroplasticity after ischemic stroke in mice. Sci Rep, 2017. 7(1): p. 6962.

72. Vibhuti, et al., Intra-arterial transplantation of human bone marrow mesenchymal stem cells (hBMMSCs) improves behavioral deficits and alters gene expression in rodent stroke model. J Neurochem, 2017. 143(6): p. 722-735.

73. Yu, X., et al., Bone marrow mesenchymal stromal cells alleviate brain white matter injury via the enhanced proliferation of oligodendrocyte progenitor cells in focal cerebral ischemic rats. Brain Res, 2018. 1680: p. 127-136.

74. Bhatia, V., et al., Randomized Assessment of the Safety and Efficacy of Intra-Arterial Infusion of Autologous Stem Cells in Subacute Ischemic Stroke. AJNR Am J Neuroradiol, 2018. 39(5): p. 899-904.

75. Chau, M.J., et al., Delayed and repeated intranasal delivery of bone marrow stromal cells increases regeneration and functional recovery after ischemic stroke in mice. BMC Neurosci, 2018. 19(1): p. 20.

76. Li, X., et al., Intravenously Delivered Allogeneic Mesenchymal Stem Cells Bidirectionally Regulate Inflammation and Induce Neurotrophic Effects in Distal Middle Cerebral Artery Occlusion Rats Within the First 7 Days After Stroke. Cell Physiol Biochem, 2018. 46(5): p. 1951-1970.

77. Bi, M., et al., Bone mesenchymal stem cells transplantation combined with mild hypothermia improves the prognosis of cerebral ischemia in rats. PLoS One, 2018. 13(8): p. e0197405.

78. Ito, Y., et al., Angiogenesis therapy for brain infarction using a slow-releasing drug delivery system for fibroblast growth factor 2. Biochem Biophys Res Commun, 2013. 432(1): p. 182-7.

79. Kawada, H., et al., Administration of hematopoietic cytokines in the subacute phase after cerebral infarction is effective for functional recovery facilitating proliferation of intrinsic neural stem/progenitor cells and transition of bone marrow-derived neuronal cells. Circulation, 2006. 113(5): p. 701-10.

80. Shichinohe, H., et al., Improved expression of gamma-aminobutyric acid receptor in mice with cerebral infarct and transplanted bone marrow stromal cells: an autoradiographic and histologic analysis. J Nucl Med, 2006. 47(3): p. 486-91.

81. Yoo, S.W., et al., Mesenchymal stem cells promote proliferation of endogenous neural stem cells and survival of newborn cells in a rat stroke model. Exp Mol Med, 2008. 40(4): p. 387-97.

82. Thored, P., et al., Long-term accumulation of microglia with proneurogenic phenotype concomitant with persistent neurogenesis in adult subventricular zone after stroke. Glia, 2009. 57(8): p. 835-49.

83. Bao, X., et al., Transplantation of human bone marrow-derived mesenchymal stem cells promotes behavioral recovery and endogenous neurogenesis after cerebral ischemia in rats. Brain Res, 2011. 1367: p. 103-13.

84. Courties, G., et al., Ischemic stroke activates hematopoietic bone marrow stem cells. Circ Res, 2015. 116(3): p. 407-17.

85. Uwamori, H., et al., Integration of neurogenesis and angiogenesis models for constructing a neurovascular tissue. Sci Rep, 2017. 7(1): p. 17349.

86. Shen, Q., et al., Endothelial cells stimulate self-renewal and expand neurogenesis of neural stem cells. Science, 2004. 304(5675): p. 1338-40.

87. Tonchev, A.B., et al., Expression of angiogenic and neurotrophic factors in the progenitor cell niche of adult monkey subventricular zone. Neuroscience, 2007. 144(4): p. 1425-35.

88. Plane, J.M., et al., Intact and injured endothelial cells differentially modulate postnatal murine forebrain neural stem cells. Neurobiol Dis, 2010. 37(1): p. 218-27.

89. Rosa, A.I., et al., The angiogenic factor angiopoietin-1 is a proneurogenic peptide on subventricular zone stem/progenitor cells. J Neurosci, 2010. 30(13): p. 4573-84.

90. Guo, S., et al., Neuroprotection via matrix-trophic coupling between cerebral endothelial cells and neurons. Proc Natl Acad Sci USA, 2008. 105(21): p. 7582-7.

91. Chen, Z., et al., Effects of Treadmill Exercise on Motor and Cognitive Function Recovery of MCAO Mice Through the Caveolin-⅟VEGF Signaling Pathway in Ischemic Penumbra. Neurochem Res, 2019. 44(4): p. 930-946.

92. Zhou, J., et al., Lactate potentiates angiogenesis and neurogenesis in experimental intracerebral hemorrhage. Exp Mol Med, 2018. 50(7): p. 78.

93. Chen, D., et al., Pyruvate Kinase M2 Increases Angiogenesis, Neurogenesis, and Functional Recovery Mediated by Upregulation of STAT3 and Focal Adhesion Kinase Activities After Ischemic Stroke in Adult Mice. Neurotherapeutics, 2018. 15(3): p. 770-784.

94. Xiao, W., et al., Elevated serum vascular endothelial growth factor in treatment-resistant schizophrenia treated with electroconvulsive therapy: Positive association with therapeutic effects. World J Biol Psychiatry, 2019. 20(2): p. 150-158.

95. Li, C., et al., Post-stroke Constraint-induced Movement Therapy Increases Functional Recovery, Angiogenesis, and Neurogenesis with Enhanced Expression of HIF-1alpha and VEGF. Curr Neurovasc Res, 2017. 14(4): p. 368-377.

96. Doeppner, T.R., et al., Conditioned Medium Derived from Neural Progenitor Cells Induces Long-term Post-ischemic Neuroprotection, Sustained Neurological Recovery, Neurogenesis, and Angiogenesis. Mol Neurobiol, 2017. 54(2): p. 1531-1540.

97. Park, H.W., et al., Human umbilical cord blood-derived mesenchymal stem cells improve functional recovery through thrombospondinl, pantraxin3, and vascular endothelial growth factor in the ischemic rat brain. J Neurosci Res, 2015. 93(12): p. 1814-25.

98. Ruan, L., et al., Coupling of neurogenesis and angiogenesis after ischemic stroke. Brain Res, 2015. 1623: p. 166-73.

99. Carmeliet, P. and C. Ruiz de Almodovar, VEGF ligands and receptors: implications in neurodevelopment and neurodegeneration. Cell Mol Life Sci, 2013. 70(10): p. 1763-78.

100. Thomas, J.L. and A. Eichmann, The power of VEGF (vascular endothelial growth factor) family molecules. Cell Mol Life Sci, 2013. 70(10): p. 1673-4.

101. Calvo, C.F., et al., Vascular endothelial growth factor receptor 3 directly regulates murine neurogenesis. Genes Dev, 2011. 25(8): p. 831-44.

102. Licht, T., et al., Reversible modulations of neuronal plasticity by VEGF. Proc Natl Acad Sci U S A, 2011. 108(12): p. 5081-6.

103. Sun, J., et al., VEGF-mediated angiogenesis stimulates neural stem cell proliferation and differentiation in the premature brain. Biochem Biophys Res Commun, 2010. 394(1): p. 146-52.

104. Hansen, T.M., A.J. Moss, and N.P. Brindle, Vascular endothelial growth factor and angiopoietins in neurovascular regeneration and protection following stroke. Curr Neurovasc Res, 2008. 5(4): p. 236-45.

105. Plaschke, K., et al., VEGF overexpression improves mice cognitive abilities after unilateral common carotid artery occlusion. Exp Neurol, 2008. 214(2): p. 285-92.

106. Wang, L., et al., Neural progenitor cells treated with EPO induce angiogenesis through the production of VEGF. J Cereb Blood Flow Metab, 2008. 28(7): p. 1361-8.

107. Wang, Y.Q., et al., VEGF overexpression enhances striatal neurogenesis in brain of adult rat after a transient middle cerebral artery occlusion. J Neurosci Res, 2007. 85(1): p. 73-82.

108. Sun, Y., et al., VEGF-induced neuroprotection, neurogenesis, and angiogenesis after focal cerebral ischemia. J Clin Invest, 2003. 111(12): p. 1843-51.

109. Jin, K., et al., Vascular endothelial growth factor (VEGF) stimulates neurogenesis in vitro and in vivo. Proc Natl Acad Sci USA, 2002. 99(18): p. 11946-50.

110. Siqueira, M., et al., Radial Glia Cells Control Angiogenesis in the Developing Cerebral Cortex Through TGF-beta1 Signaling. Mol Neurobiol, 2018. 55(5): p. 3660-3675.

111. Meng, Z., et al., Ectopic expression of human angiopoietin-1 promotes functional recovery and neurogenesis after focal cerebral ischemia. Neuroscience, 2014. 267: p. 135-46.

112. Bai, Y., et al., Ectopic expression of angiopoietin-1 promotes neuronal differentiation in neural progenitor cells through the Akt pathway. Biochem Biophys Res Commun, 2009. 378(2): p. 296-301.

113. Pringle, N.P., et al., PDGF receptors in the rat CNS: during late neurogenesis, PDGF alpha-receptor expression appears to be restricted to glial cells of the oligodendrocyte lineage. Development, 1992. 115(2): p. 535-51.

114. Jin, K., et al., Enhanced neurogenesis in Alzheimer’s disease transgenic (PDGF-APPSw,Ind) mice. Proc Natl Acad Sci USA, 2004. 101(36): p. 13363-7.

115. Mohapel, P., et al., Platelet-derived growth factor (PDGF-BB) and brain-derived neurotrophic factor (BDNF) induce striatal neurogenesis in adult rats with 6-hydroxydopamine lesions. Neuroscience, 2005. 132(3): p. 767-76.

116. Jackson, E.L., et al., PDGFR alpha-positive B cells are neural stem cells in the adult SVZ that form glioma-like growths in response to increased PDGF signaling. Neuron, 2006. 51(2): p. 187-99.

117. Funa, K. and M. Sasahara, The roles of PDGF in development and during neurogenesis in the normal and diseased nervous system. J Neuroimmune Pharmacol, 2014. 9(2): p. 168-81.

118. Moore, L., et al., PDGF-responsive progenitors persist in the subventricular zone across the lifespan. ASN Neuro, 2014. 6(2).

119. Sil, S., et al., PDGF/PDGFR axis in the neural systems. Mol Aspects Med, 2018. 62: p. 63-74.

120. Chen, J., et al., Endothelial nitric oxide synthase regulates brain-derived neurotrophic factor expression and neurogenesis after stroke in mice. J Neurosci, 2005. 25(9): p. 2366-75.

121. Madri, J.A., Modeling the neurovascular niche: implications for recovery from CNS injury. J Physiol Pharmacol, 2009. 60 Suppl 4: p. 95-104.

122. Wei, L., et al., Transplantation of hypoxia preconditioned bone marrow mesenchymal stem cells enhances angiogenesis and neurogenesis after cerebral ischemia in rats. Neurobiol Dis, 2012. 46(3): p. 635-45.

123. Pikula, A., et al., Serum brain-derived neurotrophic factor and vascular endothelial growth factor levels are associated with risk of stroke and vascular brain injury: Framingham Study. Stroke, 2013. 44(10): p. 2768-75.

124. Lin, C.Y., et al., Brain-derived neurotrophic factor increases vascular endothelial growth factor expression and enhances angiogenesis in human chondrosarcoma cells. Biochem Pharmacol, 2014. 91(4): p. 522-33.

125. Lu, L., et al., Growth Differentiation Factor 11 Promotes Neurovascular Recovery After Stroke in Mice. Front Cell Neurosci, 2018. 12: p. 205.

126. Schafer, M.J. and N.K. LeBrasseur, The influence of GDF11 on brain fate and function. Geroscience, 2019. 41(1): p. 1-11.

127. Toh, H., E. Daniels, and A. Bateman, Methods to Investigate the Roles of Progranulin in Angiogenesis Using In Vitro Strategies and Transgenic Mouse Models. Methods Mol Biol, 2018. 1806: p. 329-360.

128. Bang, O.Y., et al., Autologous mesenchymal stem cell transplantation in stroke patients. Ann Neurol, 2005. 57(6): p. 874-82.

129. Lee, J.S., et al., A long-term follow-up study of intravenous autologous mesenchymal stem cell transplantation in patients with ischemic stroke. Stem Cells, 2010. 28(6): p. 1099-106.

130. Bhasin, A., et al., Autologous mesenchymal stem cells in chronic stroke. Cerebrovasc Dis Extra, 2011. 1(1): p. 93-104.

131. Honmou, O., et al., Intravenous administration of auto serum-expanded autologous mesenchymal stem cells in stroke. Brain, 2011. 134(Pt 6): p. 1790-807.

132. Han, H., et al., Intrathecal injection of human umbilical cord blood-derived mesenchymal stem cells for the treatment of basilar artery dissection: a case report. J Med Case Rep, 2011. 5: p. 562.

133. Stein-Thoeringer, C.K., et al., Lactose drives Enterococcus expansion to promote graft-versus-host disease. Science, 2019. 366(6469): p. 1143-1149.

134. Romick-Rosendale, L.E., et al., Antibiotic Exposure and Reduced Short Chain Fatty Acid Production after Hematopoietic Stem Cell Transplant. Biol Blood Marrow Transplant, 2018. 24(12): p. 2418-2424.

135. Hidaka, D., et al., The association between the incidence of intestinal graft-vs-host disease and antibiotic use after allogeneic hematopoietic stem cell transplantation. Clin Transplant, 2018. 32(9): p. e13361.

136. Kakihana, K., Fecal microbiota transplantation for acute graft-versus-host disease of the gut. Rinsho Ketsueki, 2017. 58(5): p. 499-505.

137. Varelias, A., et al., Acute graft-versus-host disease is regulated by an IL-17-sensitive microbiome. Blood, 2017. 129(15): p. 2172-2185.

138. Andermann, T.M., A. Rezvani, and A.S. Bhatt, Microbiota Manipulation With Prebiotics and Probiotics in Patients Undergoing Stem Cell Transplantation. Curr Hematol Malig Rep, 2016. 11(1): p. 19-28.

139. Severyn, C.J., R. Brewster, and T.M. Andermann, Microbiota modification in hematology: still at the bench or ready for the bedside? Blood Adv, 2019. 3(21): p. 3461-3472.

140. Lopez, P., et al., Treg-inducing membrane vesicles from Bifidobacterium bifidum LMG13195 as potential adjuvants in immunotherapy. Vaccine, 2012. 30(5): p. 825-9.

141. Jang, S.O., et al., Asthma Prevention by Lactobacillus Rhamnosus in a Mouse Model is Associated With CD4(+)CD25(+)Foxp3(+) T Cells. Allergy Asthma Immunol Res, 2012. 4(3): p. 150-6.

142. Qiu, X., et al., Faecalibacterium prausnitzii upregulates regulatory T cells and anti-inflammatory cytokines in treating TNBS-induced colitis. J Crohns Colitis, 2013. 7(11): p. e558-68.

143. Atarashi, K., et al., Treg induction by a rationally selected mixture of Clostridia strains from the human microbiota. Nature, 2013. 500(7461): p. 232-6.

144. Yoshida, T., et al., An increased number of CD4+CD25+ cells induced by an oral administration of Lactobacillus plantarum NRIC0380 are involved in antiallergic activity. Int Arch Allergy Immunol, 2013. 162(4): p. 283-9.

145. Khailova, L., et al., Lactobacillus rhamnosus GG improves outcome in experimental pseudomonas aeruginosa pneumonia: potential role of regulatory T cells. Shock, 2013. 40(6): p. 496-503.

146. Narushima, S., et al., Characterization of the 17 strains of regulatory T cell-inducing human-derived Clostridia. Gut Microbes, 2014. 5(3): p. 333-9.

147. Kim, H.J., et al., Effects of Lactobacillus rhamnosus on allergic march model by suppressing Th2, Th17, and TSLP responses via CD4(+)CD25(+)Foxp3(+) Tregs. Clin Immunol, 2014. 153(1): p. 178-86.

148. Zheng, B., et al., Bifidobacterium breve attenuates murine dextran sodium sulfate-induced colitis and increases regulatory T cell responses. PLoS One, 2014. 9(5): p. e95441.

149. Liu, Y., et al., Lactobacillus reuteri DSM 17938 differentially modulates effector memory T cells and Foxp3+ regulatory T cells in a mouse model of necrotizing enterocolitis. Am J Physiol Gastrointest Liver Physiol, 2014. 307(2): p. G177-86.

150. Zhou, D., et al., Oral administration of a select mixture of Bacillus probiotics generates Tr1 cells in weaned F4ab/acR- pigs challenged with an F4+ ETEC/VTEC/EPEC strain. Vet Res, 2015. 46: p. 95.

151. Thakur, B.K., et al., Live and heat-killed probiotic Lactobacillus casei Lbs2 protects from experimental colitis through Toll-like receptor 2-dependent induction of T-regulatory response. Int Immunopharmacol, 2016. 36: p. 39-50.

152. Wang, K., et al., Lactobacillus casei regulates differentiation of Th17/Treg cells to reduce intestinal inflammation in mice. Can J Vet Res, 2017. 81(2): p. 122-128.

153. Fu, L., et al., Lactic acid bacteria-specific induction of CD4(+)Foxp3(+)T cells ameliorates shrimp tropomyosin-induced allergic response in mice via suppression of mTOR signaling. Sci Rep, 2017. 7(1): p. 1987.

154. Savino, F., et al., Regulatory T cells and Toll-like receptor 2 and 4 mRNA expression in infants with colic treated with Lactobacillus reuteri DSM17938. Benef Microbes, 2018. 9(6): p. 917-925.

155. Albert, M.H., et al., Antigen-dependent suppression of alloresponses by Foxp3-induced regulatory T cells in transplantation. Eur J Immunol, 2005. 35(9): p. 2598-607.

156. Rezvani, K., et al., High donor FOXP3-positive regulatory T-cell (Treg) content is associated with a low risk of GVHD following HLA-matched allogeneic SCT. Blood, 2006. 108(4): p. 1291-7.

157. Zhai, Z., et al., Correlation of the CD4+CD25high T-regulatory cells in recipients and their corresponding donors to acute GVHD. Transpl Int, 2007. 20(5): p. 440-6.

158. Arimoto, K., et al., FOXP3 expression in peripheral blood rapidly recovers and lacks correlation with the occurrence of graft-versus-host disease after allogeneic stem cell transplantation. Int J Hematol, 2007. 85(2): p. 154-62.

159. Wolf, D., et al., Regulatory T-cells in the graft and the risk of acute graft-versus-host disease after allogeneic stem cell transplantation. Transplantation, 2007. 83(8): p. 1107-13.

160. Giorgini, A. and A. Noble, Blockade of chronic graft-versus-host disease by alloantigen-induced CD4+CD25+Foxp3+ regulatory T cells in nonlymphopenic hosts. J Leukoc Biol, 2007. 82(5): p. 1053-61.

161. Hasegawa, H., et al., Therapeutic effect of CXCR3-expressing regulatory T cells on liver, lung and intestinal damages in a murine acute GVHD model. Gene Ther, 2008. 15(3): p. 171-82.

162. Zhao, D., et al., In vivo-activated CD103+CD4+ regulatory T cells ameliorate ongoing chronic graft-versus-host disease. Blood, 2008. 112(5): p. 2129-38.

163. Magenau, J.M., et al., Frequency of CD4(+)CD25(hi)FOXP3(+) regulatory T cells has diagnostic and prognostic value as a biomarker for acute graft-versus-host-disease. Biol Blood Marrow Transplant, 2010. 16(7): p. 907-14.

164. Koreth, J., et al., Interleukin-2 and regulatory T cells in graft-versus-host disease. N Engl J Med, 2011. 365(22): p. 2055-66.

165. Hannon, M., et al., Infusion of clinical-grade enriched regulatory T cells delays experimental xenogeneic graft-versus-host disease. Transfusion, 2014. 54(2): p. 353-63.

166. Theil, A., et al., Adoptive transfer of allogeneic regulatory T cells into patients with chronic graft-versus-host disease. Cytotherapy, 2015. 17(4): p. 473-86.

167. Brunstein, C.G., et al., Umbilical cord blood-derived T regulatory cells to prevent GVHD: kinetics, toxicity profile, and clinical effect. Blood, 2016. 127(8): p. 1044-51.

168. Le Texier, L., et al., Autophagy-dependent regulatory T cells are critical for the control of graft-versus-host disease. JCI Insight, 2016. 1(15): p. e86850.

169. Chen, Y.B., et al., Increased Foxp3(+)Helios(+) Regulatory T Cells and Decreased Acute Graft-versus-Host Disease after Allogeneic Bone Marrow Transplantation in Patients Receiving Sirolimus and RGI-2001, an Activator of Invariant Natural Killer T Cells. Biol Blood Marrow Transplant, 2017. 23(4): p. 625-634.

170. Ranjan, S., et al., Activated protein C protects from GvHD via PAR2/PAR3 signalling in regulatory T-cells. Nat Commun, 2017. 8(1): p. 311.

171. Kasahara, H., et al., Generation of allo-antigen-specific induced Treg stabilized by vitamin C treatment and its application for prevention of acute graft versus host disease model. Int Immunol, 2017. 29(10): p. 457-469.

172. Riegel, C., et al., Efficient treatment of murine acute GvHD by in vitro expanded donor regulatory T cells. Leukemia, 2019.

173. Kim, B., et al., Protective effects of Bacillus probiotics against high-fat diet-induced metabolic disorders in mice. PLoS One, 2018. 13(12): p. e0210120.

174. Morita, Y., et al., Long-term intake of Lactobacillus paracasei KW3110 prevents age-related chronic inflammation and retinal cell loss in physiologically aged mice. Aging (Albany NY), 2018. 10(10): p. 2723-2740.

175. Kim, D.E., et al., Lactobacillus plantarum NK3 and Bifidobacterium longum NK49 Alleviate Bacterial Vaginosis and Osteoporosis in Mice by Suppressing NF-kappaB-Linked TNF-alpha Expression. J Med Food, 2019. 22(10): p. 1022-1031.

176. Niemann-Jonsson, A., et al., Increased rate of apoptosis in intimal arterial smooth muscle cells through endogenous activation of TNF receptors. Arterioscler Thromb Vasc Biol, 2001. 21(12): p. 1909-14.

177. Chandra, D., et al., Role of aldose reductase in TNF-alpha-induced apoptosis of vascular endothelial cells. Chem Biol Interact, 2003. 143-144: p. 605-12.

178. Kubica, J., et al., Combined periprocedural evaluation of CRP and TNF-alpha enhances the prediction of clinical restenosis and major adverse cardiac events in patients undergoing percutaneous coronary interventions. Int J Mol Med, 2005. 16(1): p. 173-80.

179. Burke, J.G., et al., Human nucleus pulposis can respond to a pro-inflammatory stimulus. Spine (Phila Pa 1976), 2003. 28(24): p. 2685-93.

180. Lin, Y., et al., Propionibacterium acnes Induces Intervertebral Disc Degeneration by Promoting iNOS/NO and COX-2/PGE2 Activation via the ROS-Dependent NF-kappaB Pathway. Oxid Med Cell Longev, 2018. 2018: p. 3692752.

181. Chen, Z., et al., Overview: the role of Propionibacterium acnes in nonpyogenic intervertebral discs. Int Orthop, 2016. 40(6): p. 1291-8.

182. Yuan, Y., et al., Histological Identification of Propionibacterium acnes in Nonpyogenic Degenerated Intervertebral Discs. Biomed Res Int, 2017. 2017: p. 6192935.

183. Mottin, V.H.M. and E.S. Suyenaga, An approach on the potential use of probiotics in the treatment of skin conditions: acne and atopic dermatitis. Int J Dermatol, 2018. 57(12): p. 1425-1432.

What is claimed is:

1. A method of increasing the efficacy of a fibroblast-based treatment comprising the steps of:
a) treating dysbiosis in an individual in need thereof; and
b) administering the fibroblast-based treatment to the individual, wherein treating the dysbiosis can occur prior to, at the same time as, and/or after administering the fibroblast-based treatment.

2. The method of claim 1, further comprising the steps of:
identifying the individual in need thereof; and
assessing dysbiosis in said individual.

3. The method of claim 1 or 2, wherein the fibroblast-based treatment comprises a fibroblast-based cellular treatment.

4. The method of claim 3, wherein the fibroblast-based cellular treatment comprises fibroblast cells which are allogeneic to the recipient.

5. The method of claim 3, wherein the fibroblast-based cellular treatment comprises fibroblast cells which are autologous to the recipient.

6. The method of claim 3, wherein the fibroblast-based cellular treatment comprises fibroblast cells which are xenogeneic to the recipient.

7. The method of any one of claims 3-6, wherein the fibroblast-based cellular treatment comprises fibroblast cells which are plastic adherent.

8. The method of any one of claims 3-7, wherein the fibroblast-based cellular treatment comprises fibroblast cells which possess regenerative activity.

9. The method of claim 8, wherein the regenerative activity of the fibroblasts cells includes stimulation of angiogenesis, inhibition of inflammation, enhancement of differentiation activity, suppression of Th17, enhancement of M2 and T regulatory cell generation, augmentation of tissue self-renewal, or a combination thereof.

10. The method of claim 8 or 9, wherein the fibroblast cells express CD105.

11. The method of any one of claims 8-10, wherein the fibroblast cells express CD73.

11.1. The method of any one of claims 8-10, wherein the fibroblast cells express FGF-2.

12. The method of any one of claims 8-11.1, wherein the fibroblast cells inhibit production of IL-18.

13. The method of any one of claims 8-12, wherein the fibroblast cells inhibit macrophage activation.

14. The method of any one of claims 8-13, wherein the fibroblast cells inhibit mast cell activation.

15. The method of any one of claims 8-14, wherein the fibroblast cells produce IL-10.

16. The method of any one of claims 8-15, wherein the fibroblast cells are preconditioned by treatment with oxytocin.

17. The method of claim 16, wherein the oxytocin is administered at a concentration of 1-100 IU per ml of tissue culture media.

18. The method of claim 17, wherein the oxytocin is administered at a concentration of 10 IU per ml in tissue culture media.

19. The method of claim 1 or 2, wherein the fibroblast-based treatment is a fibroblast-conditioned culture media treatment.

20. The method of claim 19, wherein the fibroblast-conditioned culture media can induce generation of CD8 cells.

21. The method of claim 19 or 20, wherein the fibroblast-conditioned culture media comprises at least about 5 ng/ml of fibroblast-produced FGF-2.

22. The method of claim 21, wherein the fibroblast-conditioned culture media comprises at least about 2 ng/ml of fibroblast-produced FGF-1.

23. The method of any one of claims 19-22, wherein the fibroblast-conditioned culture media comprises at least about 20 pg/ml of fibroblast-produced TGF-β.

24. The method of any one of claims 19-23, wherein the fibroblast-conditioned culture media comprises exosomes derived from fibroblasts.

25. The method of claim 24, wherein the exosomes express CD81.

26. The method of claim 24 or 25, wherein the exosomes express membrane-bound TNG-β.

27. The method of any one of claims 24-26, wherein the exosomes are at least about 60-200 nm in size.

28. The method of any one of claims 1-27, wherein dysbiosis is assessed by ribosomal sequencing.

29. The method of any one of claims 1-28, wherein dysbiosis comprises an abnormality in the microbiome of the host.

30. The method of claim 29, wherein the microbiome includes commensal bacteria in the gut, blood, skin, epithelial tissue, bone marrow, thymus, spleen, liver, or a combination thereof.

31. The method of any one of claims 1-30, wherein dysbiosis results from a genetic predisposition to dysbiosis.

32. The method of any one of claims 1-30, wherein dysbiosis is induced by a treatment.

33. The method of claim 32, wherein the treatment comprises antibiotics, chemotherapy, radiation therapy, immunotherapy, hormonal therapy, or a combination thereof.

34. The method of any one of claims 1-33, wherein dysbiosis is treated by administration of microbiome components from an external source.

35. The method of claim 34, wherein the microbiome components are administered by fecal transplant.

36. The method of any one of claims 1-33, wherein dysbiosis is treated by administration of prebiotics, probiotics, or a combination thereof.

37. The method of claim 36, wherein the prebiotics comprise soluble starch, yeast extract, polydextrose, polydextrose powder, lactulose, lactosucrose, raffinose, gluco-oligosaccharide, inulin, fructo-oligosaccharide, isomalto-oligosaccharide, soybean oligosaccharides, lactosucrose, xylo-oligosaccharide, chito-oligosaccharide, manno-oligosaccharide, aribino-oligosaccharide, siallyl-oligosaccharide, fuco-oligosaccharide, galacto-oligosaccharide, gentio-oligosaccharides, or a combination thereof.

38. The method of claim 36, wherein the probiotics comprise Bifidobacterium spp., Lactobacillus spp., Streptococcus thermophilia, Bacillus coagulans, Bacillus laterosporus, Pediococcus acidilactici, Saccharomyces boulardii, or a combination thereof.

39. The method of any one of claims 1-38, wherein the fibroblast-based treatment is for stroke.

40. The method of any one of claims 1-38, wherein the fibroblast-based treatment is for degenerative disc disease.

41. The method of any one of claims 1-38, wherein the fibroblast-based treatment is for spinal cord injury.

42. The method of any one of claims 1-38, wherein the fibroblast-based treatment is for liver failure.

43. The method of any one of claims 1-38, wherein the fibroblast-based treatment is for autism.

44. The method of any one of claims 1-38, wherein the fibroblast-based treatment is for chronic inflammation.

45. The method of claim 44, wherein the chronic inflammation is due to stent placement.

46. A composition for treating dysbiosis comprising prebiotics, probiotics, or a combination thereof, wherein the composition further comprises a fibroblast-based treatment.

47. The composition of claim 46, wherein the prebiotics comprise soluble starch, yeast extract, polydextrose, polydextrose powder, lactulose, lactosucrose, raffinose, gluco-oligosaccharide, inulin, fructo-oligosaccharide, isomalto-oligosaccharide, soybean oligosaccharides, lactosucrose, xylo-oligosaccharide, chito-oligosaccharide, manno-oligosaccharide, aribino-oligosaccharide, siallyl-oligosaccharide, fuco-oligosaccharide, galacto-oligosaccharide, gentio-oligosaccharides, or a combination thereof.

48. The composition of claim 46, wherein the probiotics comprise Bifidobacterium spp., Lactobacillus spp., Streptococcus thermophilia, Bacillus coagulans, Bacillus laterosporus, Pediococcus acidilactici, Saccharomyces boulardii, or a combination thereof.

49. The composition of any one of claims 46-48, wherein dysbiosis is assessed by ribosomal sequencing.

50. The composition of any one of claims 46-49, wherein dysbiosis comprises an abnormality in the microbiome of the host.

51. The composition of claim 50, wherein the microbiome includes commensal bacteria in the gut, blood, skin, epithelial tissue, bone marrow, thymus, spleen, liver, or a combination thereof.

52. The composition of any one of claims 46-51, wherein dysbiosis results from a genetic predisposition to dysbiosis.

53. The composition of any one of claim 46-51, wherein dysbiosis is induced by a treatment.

54. The composition of claim 53, wherein the treatment comprises antibiotics, chemotherapy, radiation therapy, immunotherapy, hormonal therapy, or a combination thereof.

55. The composition of any one of claims 46-54, wherein the fibroblast-based treatment is a fibroblast-based cellular treatment.

56. The composition of claim 55, wherein the prebiotics and/or probiotics are administered prior to, at the same time as, or after the fibroblast-based cellular treatment.

57. The composition of claim 55, wherein the fibroblast-based cellular treatment comprises fibroblast cells which are allogeneic to the recipient.

58. The composition of claims 55, wherein the fibroblast-based cellular treatment comprises fibroblast cells which are autologous to the recipient.

59. The composition of claims 55, wherein the fibroblast-based cellular treatment comprises fibroblast cells which are xenogeneic to the recipient.

60. The composition of any one of claims 55-59, wherein the fibroblast-based cellular treatment comprises fibroblast cells which are plastic adherent.

61. The composition of any one of claims 55-60, wherein the fibroblast-based cellular treatment comprises fibroblast cells which possess regenerative activity.

62. The composition of claim 61, wherein the regenerative activity of the fibroblasts cells includes stimulation of angiogenesis, inhibition of inflammation, enhancement of differentiation activity, suppression of Th17, enhancement of M2 and T regulatory cell generation, augmentation of tissue self-renewal, or a combination thereof.

63. The composition of claim 61 or 62, wherein the fibroblast-based cellular treatment comprises fibroblast cells which express CD105.

64. The composition of any one of claims 61-63, wherein the fibroblast-based cellular treatment comprises fibroblast cells which express CD73.

65. The composition of any one of claims 61-64, wherein the fibroblast-based cellular treatment comprises fibroblast cells which can inhibit production of IL-18.

66. The composition of any one of claims 61-65, wherein the fibroblast-based cellular treatment comprises fibroblast cells which can inhibit macrophage activation.

67. The composition of any one of claims 61-66, wherein the fibroblast-based cellular treatment comprises fibroblast cells which can inhibit mast cell activation.

68. The composition of any one of claims 61-67, wherein the fibroblast-based cellular treatment comprises fibroblast cells which produce IL-10.

69. The composition of any one of claims 61-68, wherein the fibroblast-based cellular treatment comprises fibroblast cells preconditioned by treatment with oxytocin.

70. The composition of claim 69, wherein the oxytocin is administered at a concentration of 1-100 IU per ml of tissue culture media.

71. The composition of claim 70, wherein the oxytocin is administered at a concentration of 10 IU per ml in tissue culture media.

72. The composition of any one of claims 46-54, wherein the fibroblast-based treatment is a fibroblast-conditioned culture media treatment.

73. The composition of claim 72, wherein the prebiotics and/or probiotics are is administered prior to, at the same time as, or after the fibroblast-conditioned culture media treatment.

74. The composition of claim 72 or 73, wherein the fibroblast-conditioned culture media can induce generation of CD8 cells.

75. The composition of any one of claims 72-74, wherein the fibroblast-conditioned culture media comprises at least about 5 ng/ml of fibroblast-produced FGF-1.

76. The composition of claim 75, wherein the fibroblast-conditioned culture media comprises at least about 5 ng/ml of fibroblast-produced FGF-2.

77. The composition of any one of claims 72-76, wherein the fibroblast-conditioned culture media comprises at least about 20 pg/ml of fibroblast-produced TGF-β.

78. The composition of any one of claims 72-77, wherein the fibroblast-conditioned culture media comprises exosomes derived from fibroblasts.

79. The composition of claim 78, wherein the exosomes express CD81.

80. The composition of claim 78 or 79, wherein the exosomes express membrane-bound TNG-β.

81. The composition of any one of claims 78-80, wherein the exosomes are at least about 60-200 nm in size.

82. The composition of any one of claims 46-81, wherein the fibroblast-based treatment is for stroke.

83. The composition of any one of claims 46-81, wherein the fibroblast-based treatment is for degenerative disc disease.

84. The composition of any one of claims 46-81, wherein fibroblast-based treatment is for spinal cord injury.

85. The composition of any one of claims 46-81, wherein the fibroblast-based treatment is for liver failure.

86. The composition of any one of claims 46-81, wherein the fibroblast-based treatment is for autism.

87. The composition of any one of 46-88, wherein the fibroblast-based treatment is for chronic inflammation.

88. The composition of claim 87, wherein the chronic inflammation is due to stent placement.
